(19) United States
(12) Patent Application Publication
(10) Pub. No.: US 2023/0134870 A1
(43) Pub. Date: May 4, 2023

(54) METHOD OF POSITIONING A SWEAT SENSOR DEVICE

(71) Applicant: KONINKLIJKE PHILIPS N.V., EINDHOVEN (NL)

(72) Inventors: EDUARD GERARD MARIE PELSSERS, LIMBURG (NL); RON MARTINUS LAURENTIUS VAN LIESHOUT, GELDROP (NL); MARK THOMAS JOHNSON, ARENDONK (NL); KIRAN HAMILTON DELLIMORE, UTRECHT (NL); LAURENTIA JOHANNA HUIJBREGTS, EINDHOVEN (NL)

(21) Appl. No.: 17/910,965

(22) PCT Filed: Mar. 9, 2021
(86) PCT No.: PCT/EP2021/055923

§ 371 Date: Sep. 12, 2022

(30) Foreign Application Priority Data
Mar. 10, 2020 (NL) 2025094

(51) Int. Cl. A61B 5/145; A61B 5/00

(52) U.S. Cl. CPC A61B 5/14517; A61B 5/4266

(57) ABSTRACT

Provided is a method (400) of positioning a sweat sensor device (100), the method comprising: determining (410) a first skin location (i) of a mammalian subject, which first skin location (i) contains apocrine and eccrine sweat glands; determining (420) a second skin location (ii), adjacent to the first skin location (i), and having a different sweat gland composition than the first skin location, wherein at the second skin location (ii), predominantly eccrine sweat glands are present, and wherein the determining (410, 420) of the first (i) and second (it) skin locations is achieved by detecting differences between the first (i) and second (ii) skin locations; and positioning (430) the sweat sensor device (100) such that a first part of the sweat sensor device (100) is present on the first skin location (i), while a second part of the sweat sensor device (100) is present on the second skin location (ii).

### FIELD OF THE INVENTION

This invention relates to a method of positioning a sweat sensor device.

### BACKGROUND OF THE INVENTION

Non-invasive, semi-continuous and prolonged monitoring of biomarkers that indicate health and well-being is in demand. Such biomarker monitoring may, for example, find utility in the assessment of dehydration, stress, sleep, children's health and in perioperative monitoring. Sweat is a non-obtrusively accessible bio-fluid and is a rich source of information relating to the physiology and metabolism of the subject.

Some examples of clinically relevant components of sweat are Na⁺, Cl⁻ and/or K⁺ ions to monitor dehydration, lactate as an early warning for inflammation (which is relevant to sepsis), glucose for diabetics and neonates, and cortisol in relation to sleep apnea and stress monitoring.

The development of reliable sweat sensing has, however, been hampered by several issues, in spite of clinical work showing promising results as early as the 1940s and 1950s. To date the impactful application of sweat analysis has been limited mainly to cystic fibrosis diagnostics, and drugs and alcohol abuse testing.

As summarized by Mena-Bravo and de Castro in “Sweat: A sample with limited present applications and promising future in metabolomics” J. Pharm. Biomed. Anal. 90, 139-147 (2014), it has been found that the results from sweat sensing can be highly variable, and a correlation between values determined from blood and sweat samples appears to be lacking for various biomarkers.

Efforts have been made to address these issues by bringing wearable sensors into nearly immediate contact with sweat as it emerges from the skin. An example is the wearable patch presented by Gao et al. in “Fully integrated wearable sensor arrays for multiplexed in situ perspiration analysis” Nature 529, 509-514 (2016). The patch includes a sensor array for measuring Na⁺, K⁺, glucose, lactate, and skin temperature. However, the focus of this study is on the development and integration of the sensors themselves which, whilst evidently crucial, does not address issues relating to sweat sample collection. The latter is mostly done by placing an absorbent pad with an area in the order of several cm² between the skin and the sensor. The assumption is that, providing ample sweat is produced (hence tests are done on individuals engaged in intense exercise), the pad will absorb the sweat for analysis, and newly generated sweat will refill the pad and ‘rinse away’ the old sweat. It is, however, likely that the time-dependent response of the sensor does not directly reflect the actual level of biomarkers over time because of accumulation effects. The sample collection and presentation to the published sensors may not be well-controlled so that continuous reliable sensing over a long period of time is difficult. Such patches may also not be designed to handle the tiny amounts of sweat that are produced under normal conditions, i.e. in the order of nanoliters per minute per sweat gland.

There are two types of sweat glands: apocrine and eccrine. An ongoing debate concerns a third type: the apoeccrine gland. The apocrine and eccrine glands respectively secrete specific biomarkers, referred to more generally herein as “analytes” in variable quantities. The diagnostic outcome arising from monitoring of such a sweat analyte may, for instance, depend on which gland was responsible for producing the detected analyte.

There are both anatomical and functional differences between apocrine and eccrine sweat glands. The secretory coil of the eccrine sweat glands consists of three different cell types which all play a role in production of sweat from eccrine sweat glands. One of these cell types, which can only be found in eccrine sweat glands, are the so-called “dark cells”, which are cells that contain cytoplasmic electron-dense granules known for secreting various components such as glycoproteins, metals and the antimicrobial dermcidin. Dermcidin is an antimicrobial peptide secreted only by the eccrine sweat glands and directly attacks the bacteria on our skin. Dermcidin is one of the most abundant proteins in sweat, and is therefore a suitable marker for eccrine sweat glands. In addition, several proteins and peptides, e.g. cysteine proteinases, DNAse I, lysozyme, Zn-α2-glycoprotein, cysteine-rich secretory protein-3, have been identified in eccrine sweat. Dermcidin is not expressed in apocrine sweat glands.

Apocrine sweat glands can be found at specific body locations, for instance at the axillae, areola, ear canal, eyelids, wings of the nostril, inguinal, perineal, or perianal regions. The apocrine gland secretes a translucent turbid viscous liquid onto the skin with a pH of 5 to 6.5, albeit in minute quantities. Unlike eccrine sweat glands, which secrete in a more regular manner, the apocrine glands secrete in periodic spurts. The apocrine sweat appears on the skin surface mixed with sebum, as sebaceous glands open into the same hair follicle. It is likely that the turbidity is caused by the non-dissolvable non-aqueous components, for instance the fatty acids of the sebum which are insoluble in water.

The apocrine glands have been found to be the only sweat gland type to excrete sweat containing certain analytes, including, without any limitation, apocrine secretion odor-binding proteins 1 and 2 (ASOB1 and ASOB2), certain carbohydrates, ferric ions, lipids, steroids, sialomucin (sialomucin was also found in sweat excreted by the eccrine glands, but in negligible amounts in comparison to apocrine sweat), and/or cathelicidin.

It would be desirable to establish the concentration of sweat components as excreted by the apocrine glands in the apocrine sweat.

WO 2015/143259 A1 discloses a system and method for determining a user's physical condition.

US 2015/112165 A1 discloses devices that sense sweat and are capable of providing chronological assurance.

WO 2018/057695 A1 discloses a device for sensing a biofluid.

WO 2017/070641 A1 discloses devices and methods for buffering sweat samples.

US 2003/199743 A1 discloses a test for diagnosing the existence of certain disease states or physiological conditions in mammals based on the collection and analysis of apocrine sweat.

### SUMMARY OF THE INVENTION

It is an object of the invention to provide a suitable method of positioning a sweat sensor device. The invention is defined by the independent claim. The dependent claims define advantageous embodiments.

One aspect of the invention provides a method of positioning a sweat sensor device, the method comprising:

determining a first skin location of a mammalian subject, which first skin location contains apocrine and eccrine sweat glands;

determining a second skin location, adjacent to the first skin location, and having a different sweat gland composition than the first skin location, wherein at the second skin location, predominantly eccrine sweat glands are present, and wherein the determining of the first and second skin locations is achieved by detecting differences between the first and second skin locations; and

positioning the sweat sensor device such that a first part of the sweat sensor device is present on the first skin location, while a second part of the sweat sensor device is present on the second skin location.

The sweat sensor device positioning method of the present invention is advantageously used in combination with a method as disclosed in our non-prepublished application EP 3622880 A1 of determining a corrected concentration (Cₐ) of a first analyte (a) in sweat excreted by a first sweat gland type at a first skin location (i) having the first sweat gland type and a second sweat gland type. In particular, this second sweat gland type may not excrete sweat containing the first analyte, or may excrete sweat having such a low concentration of first analyte (in comparison with the sweat excreted by the first sweat gland) that it can be neglected, or may excrete sweat at a concentration of first analyte which is prima facie significantly different from the concentration of the first analyte in the sweat excreted by the first sweat gland that the two concentrations can be easily distinguished, the method comprising: measuring a first concentration (Cₐⁱ) of the first analyte in sweat excreted at the first skin location; measuring at least one parameter of sweat excreted by the second sweat gland type at a second skin location (ii) having predominantly the second sweat gland type, and may not have the first sweat gland type; using the at least one parameter to determine a dilution factor (Dₐⁱ) which quantifies dilution of the first analyte by sweat excreted by the second sweat gland type at the first skin location; and determining the corrected concentration (Cₐ) from the first concentration (Cₐⁱ) using the dilution factor (Dₐⁱ).

Various skin locations have both sweat glands of the first sweat gland type, e.g. the apocrine gland, and the second sweat gland type, e.g. the eccrine gland. When attempting to determine the concentration of a first analyte (a) in sweat excreted by glands of the first sweat gland type only at such a (first) skin location (i), the determination is hampered by the unknown, and potentially variable, dilution of the first analyte by the sweat excreted by glands of the second sweat gland type.

The dilution effect resulting from the sweat excreted by glands of the second sweat gland type at the first skin location (i) may be quantified by measuring at least one parameter of sweat excreted by the second sweat gland type at a second skin location (ii) which has predominantly the second sweat gland type, and may not have the first sweat gland type. This is because the respective average secretion rates of glands of the second sweat gland type at the first and second skin locations may either be equal, for instance when the first and the second skin locations are relatively close together, or may at least be proportional to each other in a predictable way. Alternatively or additionally, the respective concentrations of a second analyte solely excreted by the second type of sweat gland at both the first skin location and second skin location may be equal, or proportional to each other. However, at the first skin location, the measured concentration of the second analyte is lowered due to dilution by sweat of the first sweat gland type. By measuring the concentration of the second analyte at both skin locations, the dilution at the first skin location may be determined and this also enables determination of the dilution of the first analyte at the first skin location.

This enables determination of a dilution factor (Dₐⁱ) quantifying dilution of the first analyte by sweat excreted by the second sweat gland type at the first skin location (i) using the at least one parameter of sweat excreted by the second sweat gland type at the second skin location (ii). This dilution factor (Dₐⁱ) is then used to correct a measured first concentration (q) of the first analyte for dilution by the sweat excreted by glands of the second sweat gland type at the first skin location (i).

The at least one parameter may include a flow rate of sweat from the second sweat gland type at the second skin location. Measuring the flow rate of sweat from the second sweat gland type at the second skin location may provide a relatively simple means of determining the dilution factor (Dₐⁱ). Using the flow rate to determine the dilution factor (Dₐⁱ) may comprise using a predetermined correlation between the flow rate and the dilution factor (Dₐⁱ). In practice, the predetermined correlation may be used in the form of a graph or look-up table.

The method of EP 3622880 A1 may further comprise measuring a second concentration (Cₑⁱ) of a second analyte (e) in sweat excreted at the first skin location (i). The second analyte may be in sweat excreted by the second sweat gland type, and may not be in sweat excreted by the first sweat gland type, or may be excreted with such a low concentration of second analyte in sweat (in comparison with the one from the sweat excreted by the second sweat gland) that it can be neglected or may be excreted at a concentration of second analyte in sweat which is such prima facie significantly different from the concentration of the second analyte in the sweat excreted by the second sweat gland that the two concentrations can be easily distinguished. In this embodiment, the at least one parameter includes a third concentration (Cₑⁱⁱ) of the second analyte in sweat excreted at the second skin location (ii), and using the at least one parameter to determine the dilution factor (Dₐⁱ) comprises calculating the dilution factor using the second concentration (Cₑⁱ) and the third concentration (Cₑⁱⁱ). The dilution factor (Dₐⁱ) may, for example, be calculated using the following equation:

$${D}_{a}^{i} = 1 - \frac{{C}_{e}^{i}}{{C}_{e}^{ii}} . \qquad ( Equation I )$$

The determining the corrected concentration (Cₐ) from the first concentration (Cₐⁱ) using the dilution factor (Dₐⁱ) may comprise using the following equation:

$${C}_{a} = \frac{{C}_{a}^{i}}{{D}_{a}^{i}} . \qquad ( Equation II )$$

The method may further comprise: calculating a ratio (R_{act}) between a first local activation level of glands of the second sweat gland type at the first skin location and a second local activation level of glands of the second sweat gland type at the second skin location; and generating a value using the at least one parameter and the ratio (R_{act}), wherein the using the at least one parameter to determine the dilution factor (Dₐⁱ) comprises using the value. The ratio (R_{act}) may be used to correct for any differences between the respective local sweat gland activation levels at the first (i) and second (ii) skin locations, e.g. where the first and second skin locations are relatively far apart from each other. The ratio (R_{act}) may, for instance, be calculated using the following equation:

$${R}_{act} = \frac{{SR}_{i}^{e} \cdot {GD}_{ii}^{e}}{{SR}_{ii}^{e} \cdot {GD}_{i}^{e}} ; \qquad ( Equation III )$$

wherein SRₑⁱ and SRᵢᵢ^{e} are local sweat rates for the glands of the second sweat gland type at the first (i) and second (ii) skin locations respectively, and GDᵢ^{e} and GDᵢᵢ^{e} are local densities of the glands of the second sweat gland type at the first (i) and second (ii) skin locations respectively.

According to another aspect, EP 3622880 A1 provides an apparatus for determining a corrected concentration (Cₐ) of a first analyte (a) in sweat excreted by a first sweat gland type at a first skin location (i) having the first sweat gland type and a second sweat gland type which may not excrete sweat containing the first analyte, or may excrete sweat having such a low concentration of first analyte (in comparison with the sweat excreted by the first sweat gland) that it can be neglected, or may excrete sweat at a concentration of first analyte which is prima facie significantly different from the concentration of the first analyte in the sweat excreted by the first sweat gland that the two concentrations can be easily distinguished, the apparatus comprising: a first sensor for measuring a first concentration (CD of the first analyte in sweat excreted at the first skin location; and a second sensor for measuring at least one parameter of sweat excreted by the second sweat gland type at a second skin location (ii) having predominantly the second sweat gland type, and may not have the first sweat gland type.

The apparatus may comprise a first sweat collection hole for supplying the first sensor with sweat, and a second sweat collection hole for supplying the second sensor with sweat. The first and second sweat collection holes may thus be independent of each other, thereby to enable independent sweat sampling from the respective first and second skin locations.

The distance between the respective sweat collection holes may be at least partly determined by intended sampling location(s). The distance separating the first and second sweat collection holes from each other may be, for example, at least about 1 cm, such as at least about 2 cm.

When, for example, the first and second sweat sensors are included in a single patch, the first and second sweat collection holes may be separated from each other by at least about 1 cm, preferably by at least about 2 cm.

Irrespective of whether the first and second sweat sensors are included in a single patch, the first and second collection holes may each, for example, have a maximum area of about 2 cm², such as an area of about 1 cm².

The second sensor may comprise a flow rate sensor and the at least one parameter may thus include a flow rate of sweat from the second sweat gland type at the second skin location. The flow rate sensor may provide a relatively simple means of determining the dilution factor (Dₐⁱ). The apparatus may only require the first sensor and the flow rate sensor to enable determination of the corrected concentration (Cₐ). Such an apparatus may therefore be relatively simple and inexpensive to manufacture.

The apparatus of EP 3622880 A1 may comprise a third sensor for measuring a second concentration (Cₑⁱ) of a second analyte (e) in sweat excreted at the first skin location (i). The second analyte may be in sweat excreted by the second sweat gland type and may not be in sweat excreted by the first sweat gland type or may be excreted with such a low concentration of second analyte in sweat (in comparison with the one from the sweat excreted by the second sweat gland) that it can be neglected or may be excreted at a concentration of second analyte in sweat which is such prima facie significantly different from the concentration of the second analyte in the sweat excreted by the second sweat gland that the two concentrations can be easily distinguished. In this embodiment, the second sensor comprises a detector for measuring a third concentration (Cₑⁱⁱ) of the second analyte in sweat excreted at the second skin location (ii), and the at least one parameter includes the third concentration.

The first sensor and the second sensor may be included in a single patch for attaching to the first and second skin locations when the first skin location (i) is adjacent the second location (ii). Alternatively, the first sensor may be included in a first patch for attaching to the first skin location (i) and the second sensor may be included in a second patch for attaching to the second skin location (ii).

The single patch or the pair of first and second patches may, for instance, be positioned either side of the border between the armpit area comprising apocrine glands and the adjacent area with predominantly eccrine glands. This border is relatively sharp and therefore a distance separating the sweat collection holes of 2 cm as minimum may suffice. It may be desirable to sample from a relatively flat skin area to aid positioning of the (single) patch in a suitable fashion, taking account of the muscles below the skin and that smaller people have also relatively small armpit areas. In this regard, a hole size which has a maximum area of around 1-2 cm² is practical at the armpit side. On the adjacent area such restrictions are less strict, since the area may be smoother and flatter, but also here a hole having a maximum area of 1-2 cm² may suffice.

The apparatus of EP 3622880 A1 may further include a controller configured to: use the at least one parameter to determine a dilution factor (Dₐⁱ) corresponding to dilution of the first analyte by sweat excreted by the second sweat gland type at the first skin location; and determine the corrected concentration (Cₐ) from the first concentration (Cₐⁱ) using the dilution factor (Dₐⁱ).

The apparatus may, for instance, include a user interface for displaying the corrected concentration (Cₐ) determined by the controller.

When the second sensor comprises the flow rate sensor, the controller may be configured to determine the dilution factor (Dₐⁱ) using a predetermined correlation between the flow rate and the dilution factor (Dₐⁱ). In practice, the controller may use the predetermined correlation in the form of a graph or look-up table.

When the apparatus includes the third sensor and the detector, the controller may be configured to calculate the dilution factor using the second concentration (Cₑⁱ) and the third concentration (Cₑⁱⁱ). The dilution factor (Dₐⁱ) may, for example, be calculated by the controller using the following equation:

$${D}_{a}^{i} = 1 - \frac{{C}_{e}^{i}}{{C}_{e}^{ii}} ; \qquad ( Equation I )$$

The corrected concentration (Cₐ) may be calculated by the controller from the dilution factor (Dₐⁱ) and the first concentration (Cₐⁱ) using the following equation:

$${C}_{a} = \frac{{C}_{a}^{i}}{{D}_{a}^{i}} . \qquad ( Equation II )$$

The controller may be configured to: calculate a ratio (R_{act}) between a first local activation level of glands of the second sweat gland type at the first skin location and a second local activation level of glands of the second sweat gland type at the second skin location; and generate a value using the at least one parameter and the ratio (R_{act}). In this embodiment, the controller is configured to determine the dilution factor (Dₐⁱ) using the value. The ratio (R_{act}) may be used to correct for any differences between the respective local sweat gland activation levels at the first and second skin locations, e.g. where the first and second skin locations are relatively far apart from each other. The ratio (R_{act}) may, for instance, be calculated by the controller using the following equation:

$${R}_{act} = \frac{{SR}_{i}^{e} \cdot {GD}_{ii}^{e}}{{SR}_{ii}^{e} \cdot {GD}_{i}^{e}} ; \qquad ( Equation III )$$

wherein SRᵢ^{e} and SRᵢᵢ^{e} are local sweat rates for the glands of the second sweat gland type at the first (i) and second (ii) skin locations respectively, and GDᵢ^{e} and GDᵢᵢ^{e} are local densities of the glands of the second sweat gland type at the first (i) and second (ii) skin locations respectively.

### BRIEF DESCRIPTION OF THE DRAWINGS

Embodiments of the invention are described in more detail and by way of non-limiting examples with reference to the accompanying drawings, wherein:

FIGS. 1-5 illustrate various embodiments of a sweat analyte concentration determining apparatus as disclosed in our non-prepublished application EP 3622880 A1; and

FIGS. 6-25 illustrate various embodiments of a method of the present invention of positioning a sweat sensor device, which method could be advantageously used with and in the context of the apparatuses of FIGS. 1-5. A movement direction is indicated by an arrow.

### DETAILED DESCRIPTION OF THE EMBODIMENTS

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

EP 3622880 A1 provides a differential measuring method (and a related apparatus) for determining the corrected concentration (Cₐ). The first skin location (i) may, for instance, have apocrine and eccrine glands. By measuring the first concentration (Cₐⁱ) at the first skin location (i) of the first analyte (a) solely originating from apocrine glands (although at this stage diluted to an unknown degree by the eccrine glands at the the first skin location (i)) and measuring the parameter of sweat excreted by the eccrine glands at the second skin location (ii), the undiluted concentration of the first analyte (a) in the apocrine sweat may be determined unambiguously.

There is still a debate ongoing about the existence of a third gland type in the axilla: the apoeccrine gland. For present purposes, the first sweat gland type may, for instance, be regarded as including both the apocrine and apoeccrine glands. In this case, the second sweat gland type would correspond to the eccrine gland.

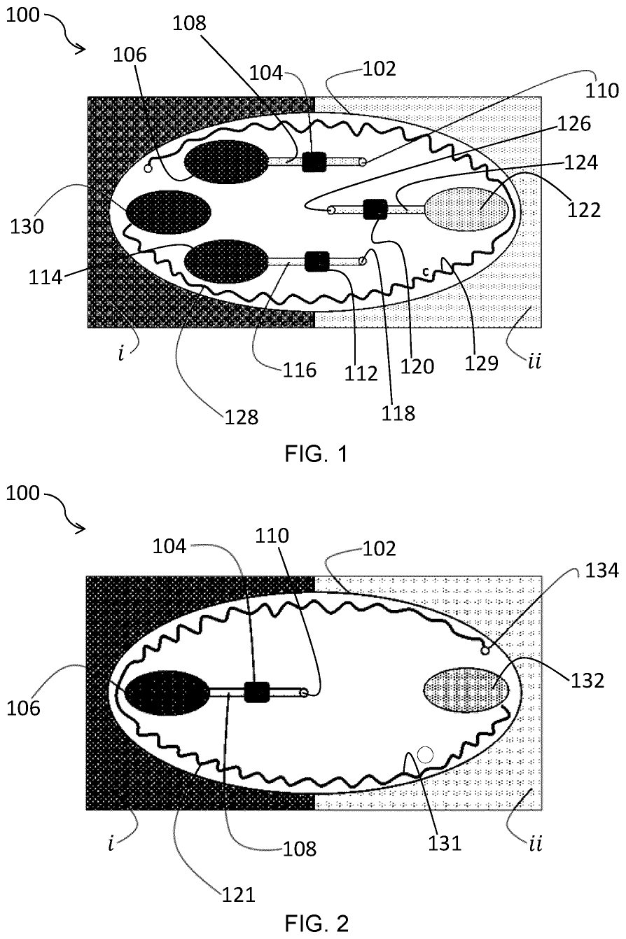

FIG. 1 shows a first embodiment of a sweat analyte concentration determining apparatus 100. The apparatus 100 comprises a single patch 102 which is positioned on, e.g. adhered to, a first skin location i, as represented by the darker pattern on the left hand side of FIG. 1, and a second skin location ii which is adjacent the first skin location i. A single patch 102 spanning the adjacent first i and second ii skin locations may mean that the respective average secretion rates of glands of the second sweat gland type at the first i and second ii skin locations may be equal, or close to equal, which may simplify calculation of the dilution factor (Dₐⁱ). Alternatively, the apparatus 100 may include two separate patches, e.g. for respectively attaching to non-adjacent first i and second ii skin locations.

Whilst not apparent from the plan view provided in FIGS. 1 and 2, the patch 102 may include a first layer which contacts the skin and a second layer disposed on the first layer, such that the first layer is effectively interposed between the skin and the second layer. The second layer may cover the various sweat sampling and sensing components of the patch 102.

As shown in FIG. 1, the apparatus 100 comprises a first sensor 104. The first sensor 104 is for measuring a first concentration (CD of the first analyte at the first skin location i. Sweat is collected from the first skin location i by a first collection hole 106 in the first layer and transported to the first sensor 104 via a channel 108. The channel 108 extends past the first sensor 104 and terminates at an air vent 110 delimited by the second layer.

The first sensor 104 may employ any suitable analyte concentration measurement principle, providing the first sensor 104 is able to measure the concentration of the first analyte (a). For example, colorimetry, electrical impedance, labelled antibodies, etc. may be used in the concentration measurement of the first analyte (a). A technique using labelled antibodies may, for instance, be used for protein concentration determination for specific proteins.

A second sensor 120, is provided in the apparatus 100 for measuring the at least one parameter of sweat excreted by the second sweat gland type at the second skin location ii, which has predominantly the second sweat gland type, and may not have the first sweat gland type.

In the embodiment shown in FIG. 1, the second sensor includes a detector 120 for measuring a (third) concentration (Cₑⁱⁱ) of the second analyte at the second skin location ii. The detector 120 is supplied with sweat by the channel 124 extending between the second collection hole 122, which receives sweat from the second skin location ii, and the detector 120. The channel 124 further extends beyond the detector 120, and terminates at the air vent 126.

The distance between the first and second sweat collection holes 106, 122 (132 in FIG. 2) may be at least partly determined by intended sampling location(s). The distance separating the first and second sweat collection holes 106, 122 (132 in FIG. 2) from each other may be, for example, at least about 1 cm, such as at least about 2 cm.

When, for example, the first sweat sensor 104 and the second sweat sensor 120, are included in a single patch, the first and second sweat collection holes 106, 122 (132 in FIG. 2) may be separated from each other, i.e. the distance between the respective edges of the sweat collection holes 106, 122 (132 in FIG. 2), by at least about 1 cm, such as at least about 2 cm.

Irrespective of whether the first and second sweat sensors 104, 120, 121 are included in a single patch, the first and second collection holes 106, 122 (132 in FIG. 2) may each, for example, have a maximum area of about 2 cm², such as an area of about 1 cm².

The detector 120 may employ any suitable analyte concentration measurement principle, providing the detector 120 is able to measure the (third) concentration (Cₑⁱⁱ) of the second analyte (e) at the second skin location ii. For example, colorimetry, electrical impedance, labelled antibodies, etc. may be used in the concentration measurement of the second analyte (e).

In the embodiment shown in FIG. 1, a third sensor 112 is provided for measuring a (second) concentration (Cₑⁱ) of a second analyte (e) at the first skin location i. The second analyte may be in sweat excreted by the second sweat gland type and may not be in sweat excreted by the first sweat gland type. The third sensor 112 is supplied with sweat by the channel 116 extending between the further first collection hole 114, which receives sweat from the first skin location i, and the third sensor 112. The channel 116 further extends beyond the third sensor 112, and terminates at the air vent 118.

The third sensor 112 may employ any suitable analyte concentration measurement principle, providing the third sensor 112 is able to measure the (second) concentration (Cₑⁱ) of the second analyte (e) at the first skin location i. For example, colorimetry, electrical impedance, labelled antibodies, etc. may be used in the concentration measurement of the second analyte (e).

An optional flow rate analyzer 128 may be included in the apparatus 100, as shown in FIG. 1. This flow rate analyzer 128 may, for instance, comprise a thin channel 129 extending around the patch 102. The thin channel 129, which is progressively filled with sweat via an additional collection hole 130 at the first skin location i, provides an indication of the flow rate from the first skin location i by measurement of the length of the thin channel 129 which becomes filled with sweat as a function of time. The term “thin” in this context (and in relation to the flow rate sensor 121) refers to the channel 129 being thinner, i.e. having a relatively smaller diameter bore, in comparison to the channels 108, 116 and 124 which are intended to carry sweat to the respective sensor/detector 104, 116 and 120, rather than providing an indication of flow rate.

Any suitable detection principle may be used to measure the degree of filling of the thin channel 129. For example, the position of the meniscus in the thin channel 129 as a function of time may be determined from a suitable image. In this respect, the flow rate analyzer 128 may include a camera (not shown), and the apparatus 100 may, for instance, include a controller (not shown in FIGS. 1 and 2) loaded with suitable image analyzing software for detecting the meniscus. Alternative flow rate sensing principles may also be contemplated, such as calorimetric flow sensing, temperature gradient driven flow sensing, etc.

Whilst the flow rate analyzer 128 may be useful due to the dependency of concentrations of particular components on the sweat rate, this flow rate analyzer 128 is not essential in the context of the embodiment shown in FIG. 1 for determining the dilution factor (Dₐⁱ) which quantifies dilution of the first analyte by sweat excreted by the second sweat gland type at the first skin location i. The dilution factor (Dₐⁱ) may be derived using the second concentration (Cₑⁱⁱ) and the third concentration (Cₑⁱⁱ), as respectively measured by the third sensor 112 and the detector 120 in the apparatus 100 as depicted in FIG. 1, as will now be explained in more detail.

The first concentration (Cₑⁱ) of the first analyte (a) at the first skin location i, as measured using the first sensor 104, may be expressed in terms of the dilution factor (Dₐⁱ) and the corrected concentration (Cₐ) in the following way:

Cₐⁱ=Cₐ·Dₐⁱ (Equation A).

Similarly, the second concentration (Cₑⁱ) of the second analyte (e) at the first skin location i, as measured using the third sensor 112, may be expressed in terms of a further dilution factor (Dₑ^{t}), which quantifies dilution of the second analyte (e) by sweat excreted by the first sweat gland type at the first skin location i, and a corrected concentration of the second analyte (Cₑ) in the following way:

Cₑⁱ=Cₑ·Dₑⁱ (Equation B).

The respective dilution factors Dₐⁱ and Dₑⁱ both have values between 0 and 1, and are related to each other by the following equation:

Dₐⁱ+Dₑⁱ=1 (Equation C).

Equation C reflects the mutual dilution of the respective sweats excreted by the first and second sweat gland types at the first skin location i. Combining Equations B and C gives:

$${D}_{a}^{i} = 1 - \frac{{C}_{e}^{i}}{{C}_{e}} . \qquad ( Equation D )$$

It may be assumed that the undiluted concentration of the second analyte (e) in sweat of the second sweat gland type only at the first skin location i (i.e. correcting for the diluting effect of the sweat from the first sweat gland type) is equal, or at least very similar, to the concentration of the second analyte (e) at the second skin location ii, i.e.

Cₑ=Cₑⁱⁱ (Equation E);

This assumption holds particularly when the first and second locations i and ii are relatively close to each other, as may be the single patch 102 embodiments depicted in FIGS. 1 and 2, providing that the patch 102 has an area, for example, in the order of only a few cm². Note that in Equation E, Cₑⁱⁱ is the (third) concentration of the second analyte (e) at the second skin location ii. Substituting Equation E in Equation D gives Equation I:

$${D}_{a}^{i} = 1 - \frac{{C}_{e}^{i}}{{C}_{e}^{ii}} ; \qquad ( Equation I )$$

Using Equation I, the second concentration (Cₑⁱ) and the third concentration (Cₑⁱⁱ) measured using the third sensor 112 and the detector 120 respectively, the dilution factor (Dₐⁱ) may thus be determined. The corrected concentration (Cₐ) may then be calculated from the dilution factor (Dₐⁱ) and the first concentration (Cₐⁱ) using Equation II (obtained by rearranging Equation A):

$${C}_{a} = \frac{{C}_{a}^{i}}{{D}_{a}^{i}} . \qquad ( Equation II )$$

The units of the corrected concentration (Cₐ), the first concentration (Cₐⁱ), the second concentration (Cₑⁱ) and the third concentration (Cₑⁱⁱ) may all be, for instance, mol/L. Whilst not essential, measuring a total sweat flow rate using the flow rate analyzer 128 may permit assessment of the mean quantity of sweat that the subject is excreting, which may be used to refine the above calculations.

Whilst not shown in FIG. 1, the apparatus 100 may optionally include a flow rate sensor for measuring a flow rate of sweat from the second sweat gland type at the second skin location ii. Such a flow rate sensor may be an alternative or in addition to the flow rate analyzer 128. Such a flow rate sensor may, as will be described in more detail in relation to FIG. 2, provide an alternative means for estimating the dilution factor (Dₐⁱ). This may, for example, provide verification or enable refinement of the dilution factor (Dₐⁱ) derived from measuring the second Cₑⁱ and third Cₑⁱⁱ concentrations of the second analyte (e).

FIG. 2 illustrates a second embodiment of a sweat analyte concentration determining apparatus 100. As in the case of FIG. 1, the apparatus 100 comprises a single patch 102 which is positioned on, e.g. adhered to, the first skin location i, as represented by the darker pattern on the left side of the apparatus 100, and a second skin location ii which is adjacent the first skin location i. Alternatively, the apparatus 100 may include two separate patches, e.g. for respectively attaching to non-adjacent first i and second ii skin locations.

As shown in FIG. 2, the apparatus 100 comprises a first sensor 104 for measuring a first concentration (Cₐⁱ) of the first analyte at the first skin location i. Similarly to the embodiment shown in FIG. 1, in operation of the apparatus 100 depicted in FIG. 2 sweat is collected from the first skin location i by a first collection hole 106 in the first layer of the patch 102 and transported to the sensor 104 via a channel 108. The channel 108 extends past the first sensor 104 and terminates at an air vent 110 delimited by the second layer of the patch 102.

The first sensor 104 may employ any suitable analyte concentration measurement principle providing the first sensor 104 is able to measure the concentration of the first analyte (a). For example, colorimetry, electrical impedance, or labelled antibodies, etc. may be used in the concentration measurement of the first analyte (a).

In the embodiment shown in FIG. 2, the second sensor for measuring the at least one parameter of sweat excreted by the second sweat gland type at the second skin location ii comprises a flow rate sensor 121. This flow rate sensor 121 may, for instance, comprise a thin channel 131 extending around the patch 102. The thin channel 131 is interposed between the first and second layers of the patch 102, and terminates at an air vent 134 which corresponds to an aperture delimited by the second layer. The thin channel 131 is progressively filled with sweat via a second collection hole 132 at the second skin location ii, and thus provides an indication of the flow rate from the second skin location ii by measurement of the length of the thin channel 131 which becomes filled with sweat as a function of time.

Any suitable detection principle may be used to measure the degree of filling of the thin channel 131. For example, the position of the meniscus in the thin channel 131 as a function of time may be determined from a suitable image. In this respect, the flow rate sensor 121 may include a camera (not shown), and the apparatus 100 may include a controller (not shown in FIGS. 1 and 2) loaded with suitable image analyzing software. Alternative flow rate sensing principles may also be contemplated, such as calorimetric flow sensing, temperature gradient driven flow sensing, etc. Such flow rate sensing principles are well-known per se and will not be further described herein for the sake of brevity only.

The inclusion of the flow rate sensor 121 in the apparatus 100 shown in FIG. 2 means that the at least one parameter may include a flow rate of sweat from the second sweat gland type at the second skin location ii. The dilution factor Dₐⁱ may be determined from this measured flow rate.

In other words, the measured flow rate of sweat from the second sweat gland type at the second skin location ii may be used to derive, via the dilution factor Dₐⁱ, the real concentration of the first analyte (e.g. solely secreted by the apocrine gland) in the sweat excreted by the first sweat gland (e.g. apocrine sweat) at the first skin location i.

In an embodiment, determining the dilution factor (Dₐⁱ) from the flow rate of sweat from the second sweat gland type at the second skin location ii comprises using a predetermined correlation between the flow rate and the dilution factor (Dₐⁱ).

In order to attain such a predetermined correlation, a set of volunteers may, for instance, be used. Since these persons will have variable flow rates from glands of the second sweat gland type (e.g. eccrine glands), a correlation may be made of the dilution factor (Dₐⁱ) as function of the flow rate of sweat from the second sweat gland type (e.g. eccrine glands) at the second skin location ii.

The apparatus 100 shown in FIG. 1 may, for example, be used to determine the dilution factors (Dₐⁱ) of each of the volunteers by measuring the second (Cₑⁱ) and third (Cₑⁱⁱ) concentrations of the second analyte (e) at the first i and second ii skin locations respectively, as previously described. A suitable flow rate sensor, such as the flow rate sensor 121 described above in relation to the apparatus 100 shown in FIG. 2 may be used to determine the flow rate of sweat from the second sweat gland type at the second skin location ii for each of the volunteers.

A correlation may thus be made between the dilution factor (Dₐⁱ) and the flow rate of sweat from the second sweat gland type at the second skin location ii using the data from the volunteers. The resulting (predetermined) correlation may be, for example, in the form of a look-up table or graph, which may then be used determine the dilution factor (Dₐⁱ) for a flow rate measured using the flow rate sensor 121 of the apparatus 100 shown in FIG. 2. In turn, the determined dilution factor (Dₐⁱ) may then be used to determine the corrected concentration (Cₐ) from the first concentration (Cₐⁱ). The predetermined correlation may thus effectively permit extrapolation to zero flow rate from the second sweat gland type (e.g. eccrine glands) at the first skin location i such that the corrected concentration (Cₐ) may be determined.

Whilst use of such volunteer data may lead to lower accuracy for an individual, in certain clinical applications the accuracy may be sufficient. Moreover, in the embodiment shown in FIG. 2, only two sensors are required which may make the apparatus 100 simpler and cheaper to produce. On the other hand, additional concentration sensors, such as the third sensor 112 and the detector 120, as described in relation to the embodiment shown in FIG. 1, and/or further flow rate analyzers may optionally be included in the apparatus 100 shown in FIG. 2.

At this point it is noted that the connections to and from the various sensors and detectors in the apparatuses 100 depicted in FIGS. 1 and 2 are not shown for the sake of clarity. These connections may, for instance, include wires for providing power to the sensors and/or for communicating the sensor/detector signals to a controller (not shown in FIGS. 1 and 2) which records/displays the signals via a suitably configured user interface (not shown in FIGS. 1 and 2). Alternatively or additionally, the patch (or patches) 102 may include an on-board chip with an antenna which can receive power wirelessly, and/or transmit the sensor/detector signals wirelessly to the controller recording the signal and/or providing power to the sensors/detectors.

Additional sensors may also be included in the apparatuses 100 shown in FIGS. 1 and 2, which additional sensors may be employed to measure other components originating from the first sweat gland type, i.e. in addition to the first analyte (a). The same techniques as explained above may be used to correct for the dilution due to sweat from glands of the second sweat gland type at the first skin location i.

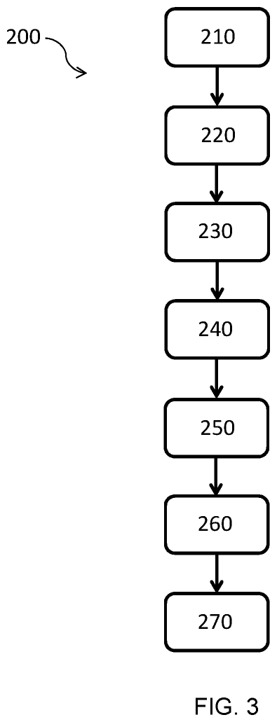

FIG. 3 shows a flowchart of a sweat analyte concentration determining method 200. In step 210, a first concentration (Cₐⁱ) of the first analyte at a first skin location (i) is measured. This may be achieved using the first sensor 104 of the apparatus 100, as previously described.

In step 220, at least one parameter is measured. The at least one parameter relates to sweat excreted by the second sweat gland type at a second skin location (ii) having predominantly the second sweat gland type and may not have the first sweat gland type. The at least one parameter is then used in step 260 to determine a dilution factor (Dₐⁱ) which quantifies dilution of the first analyte by sweat excreted by the second sweat gland type at the first skin location (i). In step 270, the first concentration (Cₐⁱ) is corrected using the dilution factor (Dₐⁱ) so as to provide a corrected concentration (Cₐ) of the first analyte (a).

Measuring 220 the at least one parameter may include measuring a flow rate of sweat from the second sweat gland type at the second skin location. This may be achieved, for instance, using the flow rate sensor 121 of the apparatus 100 shown in FIG. 2. In such a scenario, the using 260 the at least one parameter to determine the dilution factor (Dₐⁱ) may comprise using a predetermined correlation between the flow rate and the dilution factor (Dₐⁱ), as previously described.

Alternatively or additionally, the method 200 may further comprise measuring 230 a second concentration (Cₑⁱ) of a second analyte (e) in sweat excreted by the second sweat gland type at the first skin location (i). This may, for instance, be achieved using the third sensor 112 of the apparatus 100 shown in FIG. 1. In such an embodiment, measuring 220 the at least one parameter includes measuring a third concentration (Cₑⁱⁱ) of the second analyte at the second skin location (ii), which may be achieved using the detector 120 of the apparatus 100 shown in FIG. 1.

Using 260 the at least one parameter to determine the dilution factor (Dₐⁱ) may comprise calculating the dilution factor (Dₐⁱ) using the second concentration (Cₑⁱ) and the third concentration (Cₑⁱⁱ), e.g. using Equation I, as previously described. Determining 270 the corrected concentration (Cₐ) from the first concentration (Cₐⁱ) using the dilution factor (Dₐⁱ) may use Equation II.

The method 200 may further include steps which enable anatomical variations in sweat gland density and sweat gland activation levels to be accounted for. Whilst the apparatuses 100 shown in FIGS. 1 and 2 include a single patch 102, this is not intended to be limiting. Alternatively, a first patch may be attached to the first skin location (i) and a second patch may be attached to the second skin location (ii). In such an embodiment, the first sensor 104 is included in the first patch and the second sensor 120, 121 is included in the second patch.

It may be a reasonable assumption that the average secretion rate per gland of the second sweat gland type (e.g. eccrine gland) is equal for nearby skin locations, e.g. spanned by the same patch 102. However, when the two skin locations (i) and (ii) are relatively far apart from each other, the average secretion rates of the second sweat gland type at the respective skin locations may usefully be taken into account.

It has been shown in previous studies, such as by Taylor and Machado-Moreira in “Regional variations in transepidermal water loss, eccrine sweat gland density, sweat secretion rates and electrolyte composition in resting and exercising humans” Extreme Physiology & Medicine 2013; 2:4 (referred to herein below simply as “Taylor”), that although sweating is synchronous across the entire body, eccrine glands from different regions of the body may discharge sweat at different rates. This may in turn imply that there may be a difference in biomarker concentration in the secreted sweat at different regions of the body, which is likely due to anatomical and physiological variations. According to Kondo et al. in “Regional difference in the effect of exercise intensity on thermoregulatory sweating and cutaneous vasodilation” Acta Physiologica Scandinavica 1998, 164:71-78, the level of sweat gland activation can vary between different skin regions with the sweat rate determined by both glandular recruitment and increases in flow rate.

The method 200 may therefore include the following additional steps, which account for regional variations in both the sweat gland density and the sweat gland secretion/discharge rate.

In step 240, a ratio (R_{act}) between a first local activation level of glands of the second sweat gland type at the first skin location and a second local activation level of glands of the second sweat gland type at the second skin location may be calculated. In step 250, a value is generated using the at least one parameter and the ratio (R_{act}). In this case, the value is used in step 260 to determine the dilution factor (Dₐⁱ).

In an embodiment, the ratio (R_{act}) is calculated using the following equation:

$${R}_{act} = \frac{{SR}_{i}^{e} \cdot {GD}_{ii}^{e}}{{SR}_{ii}^{e} \cdot {GD}_{i}^{e}} ; \qquad ( Equation III )$$

wherein SRᵢ^{e} and SRᵢᵢ^{e} are local sweat rates for the glands of the second sweat gland type at the first (i) and second (ii) skin locations respectively, and GDᵢ^{e} and GDᵢᵢ^{e} are local densities of the glands of the second sweat gland type at the first (i) and second (ii) skin locations respectively.

Equation III may be derived in the following way. The local sweat rate of the second sweat gland type at a given location (SR_{loc}^{e}) may be expressed as:

SR_{loc}^{e}=SR_{ag}^{e}·N_{ag}^{e} (Equation F);

wherein SR_{ag}^{e} is the average sweat rate per activated gland of the second sweat gland type and N_{ag}^{e} is the average number of activated glands of the second sweat gland type.

N_{ag}^{e}=r_{loc}^{e}·GD_{loc}^{e}·A_{patch} (Equation G);

wherein r_{loc}^{e} is the local ratio of active to inactive glands of the second sweat gland type, GD_{loc}^{e} is the local sweat gland density of glands of the second sweat gland type (which can, for instance, be derived from Taylor (see Table 3 of Taylor)) and A_{patch} is the patch area (which is a known quantity).

The sweat rate is measured at two different skin locations (i) and (ii), yielding two different sweat rates, SRᵢ^{e} and SRᵢᵢ^{e}, respectively:

SRᵢ^{e}=SR_{ag;i}^{e}·N_{ag;i}^{e}=SR_{ag;i}^{e}·rᵢ^{e}·GDᵢ^{e}·A_{patch;i} (Equation H);

SRᵢᵢ^{e}=AR_{ag;ii}^{e}·=SR_{ag;ii}^{e}·GDᵢᵢ^{e}·A_{patch;ii} (Equation H);

Rearranging Equations H and J gives:

$${SR}_{ag ; i}^{e} \cdot {r}_{i}^{e} = \frac{{SR}_{i}^{e}}{{GD}_{i}^{e} \cdot {A}_{patch ; i}} ; \qquad ( Equation K )$$

$${SR}_{ag ; ii}^{e} \cdot {r}_{ii}^{e} = \frac{{SR}_{ii}^{e}}{{GD}_{ii}^{e} \cdot {A}_{patch ; ii}} . \qquad ( Equation L )$$

Dividing Equation K by Equation L gives:

$$\frac{{SR}_{ag ; i}^{e} \cdot {r}_{i}^{e}}{{SR}_{ag ; ii}^{e} \cdot {r}_{ii}^{e}} = \frac{{SR}_{i}^{e}}{{SR}_{ii}^{e}} \cdot \frac{{GD}_{ii}^{e} \cdot {A}_{patch ; ii}}{{GD}_{i}^{e} \cdot {A}_{patch ; i}} ; \qquad ( Equation M )$$

Assuming for simplicity that A_{patch; ii}=A_{patch;i} (i.e. the patch areas at the two skin locations are the same) and grouping SR_{ag;i}^{e}·rᵢ^{e}/SR_{ag;ii}^{e}·rᵢᵢ^{e} into a single term, R_{act}, which captures the ratio of the local activation level of the sweat glands at the two sites, Equation M simplifies to:

$${R}_{act} = \frac{{SR}_{i}^{e}}{{SR}_{ii}^{e}} \cdot \frac{{GD}_{ii}^{e}}{{GD}_{i}^{e}} . \qquad ( Equation III )$$

If rᵢ^{e} is assumed to equal rᵢᵢ^{e} (i.e. the ratio of active to inactive sweat glands at the two sites is the same) then the ratio R_{act} of sweat gland activity

$$( \frac{{SR}_{ag ; i}^{e}}{{SR}_{ag ; ii}^{e}} )$$

at the first and second skin locations may be estimated.

The ratio R_{act} may be used, for instance, to correct the measured (third) concentration of the second analyte (Cₑⁱⁱ) at the second skin location (ii), for situations where the assumption that Cₑ=Cₑⁱⁱ (Equation E) may be less applicable, e.g. where the first and second patches are relatively far apart from each other. In such a scenario, the measured (third) concentration Cₑⁱⁱ may be corrected by multiplying by R_{act}. The resulting value may then be used in determining the dilution factor (Dₐⁱ) using Equation I.

Alternatively, the ratio R_{act} may be used to correct the measured (second) concentration of the second analyte (Cₑⁱ) at the first skin location (i), in which case the (second) concentration Cₑⁱ may be corrected by multiplying by

$$\frac{1}{{R}_{act}} .$$

To implement step 240, the known (average) anatomical second sweat gland density (e.g. eccrine; see Table 3 provided in Taylor) may be used, together with a suitable correlation, e.g. a look-up table, of sweat gland discharge rate with the local sweat rate, the sweat gland density and the sweat gland activity. Such a correlation may be attained from volunteer testing.

In a non-limiting example, when the first and second patches are placed on the forehead and dorsal foot respectively, R_{act} at the peak sweat rate would be, using Equation III:

$${R}_{act} = ( 2.5 ) * ( \frac{119}{186} ) = 1.6$$

The ratio 119/186 was derived from Table 3 provided in Taylor, and the number 2.5 for the ratio

$$\frac{{SR}_{i}^{e}}{{SR}_{ii}^{e}}$$

has been derived from the graphs shown in FIG. 3 of Taylor. The latter ratio was determined at the peak local sweat rate at 16 minutes for the forehead and for the dorsal foot. Dividing the respective peak heights for the forehead and the dorsal foot gives 2.5.

R_{act}=1.6 implies that the sweat gland activity level at the forehead is 1.6 times that at the dorsal foot location. This may suggest that concentration of the second analyte at the forehead is therefore 1.6 times higher than at the dorsal foot.

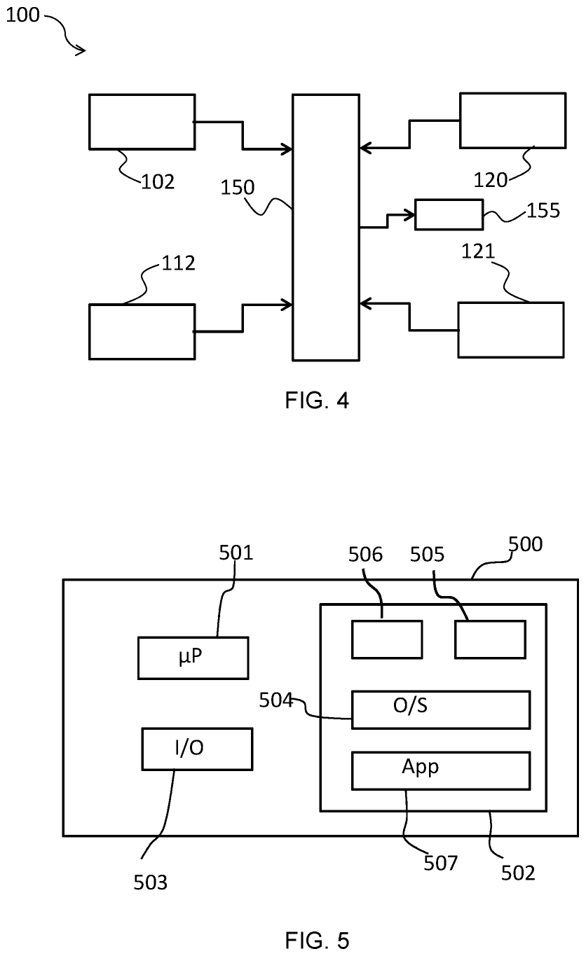

FIG. 4 shows a block diagram of another embodiment of a sweat analyte concentration determining apparatus 100. The apparatus 100 includes the first sensor 104 and the second sensor 120, 121, and a controller 150. The controller 150 receives information from the various sensors/detectors included in the apparatus 100, as shown by the arrows pointing from the sensors/detectors to the controller 150. This information may be communicated to the controller 150 via wires or wirelessly, as previously described.

In this embodiment, the controller 150 uses the at least one parameter measured by the second sensor 120, 121 to determine the dilution factor (Dₐⁱ). The controller 150 then determines the corrected concentration (Cₐ) from the first concentration (Cₐⁱ) using the dilution factor (Dₐⁱ). In other words, the controller 150 is configured to implement steps 260 and 270 of the method 200 described above.

When the second sensor comprises the flow rate sensor 121, the controller 150 may determine the dilution factor (Dₐⁱ) using the predetermined correlation between the flow rate and the dilution factor (Dₐⁱ). The controller 150 may also be configured to detect the meniscus of the sweat in the thin channel 131 from a suitable image, i.e. during the process of determining the flow rate, as previously described.

Alternatively or additionally, when the apparatus 100 includes the third sensor 112 and the detector 120, the controller 150 may determine the dilution factor (Dₐⁱ) using the second concentration (Cₑⁱ) and the third concentration (Cₑⁱⁱ), e.g. using Equation I. The corrected concentration (Cₐ) may then be calculated from the dilution factor (Dₐⁱ) and the first concentration (Cₐⁱ) using Equation II, as previously described.

In an embodiment, the controller 150 is further configured to implement steps 240 and 250 of the method 200. In this respect, the controller 150 may calculate the ratio (R_{act}) between the first local activation level of glands of the second sweat gland type at the first skin location and the second local activation level of glands of the second sweat gland type at the second skin location, e.g. using Equation III. The controller 150 may then generate the value using the at least one parameter and the ratio (R_{act}), and determine the dilution factor (Dₐⁱ) using the value.

As shown in FIG. 4, the apparatus 100 includes a user interface 155. As shown by the arrow pointing from the controller 150 to the user interface 155, information received and/or computed by the controller 150 may be sent to the user interface 155, which may then display the information. In particular, the user interface 155 may be used to display the corrected concentration (Cₐ) of the first analyte (a) determined by the controller 150. The user interface 155 may include any suitable display type. For example, the user interface 155 may include a LED/LCD display, which may have touchscreen capability permitting entry of parameters by the user, e.g. sweat rate and/or sweat gland density values for use in the R_{act} calculation, and so on.

FIG. 5 illustrates an example of a computer 500 for implementing the controller 150 described above.

The computer 500 includes, but is not limited to, PCs, workstations, laptops, PDAs, palm devices, servers, storages, and the like. Generally, in terms of hardware architecture, the computer 500 may include one or more processors 501, memory 502, and one or more I/O devices 503 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 501 is a hardware device for executing software that can be stored in the memory 502. The processor 501 can be virtually any custom made or commercially available processor, a central processing unit (CPU), a digital signal processor (DSP), or an auxiliary processor among several processors associated with the computer 500, and the processor 501 may be a semiconductor based microprocessor (in the form of a microchip) or a microprocessor.

The memory 502 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 502 may incorporate electronic, magnetic, optical, and/or other types of storage media. Note that the memory 502 can have a distributed architecture, where various components are situated remote from one another, but can be accessed by the processor 501.

The software in the memory 502 may include one or more separate programs, each of which comprises an ordered listing of executable instructions for implementing logical functions. The software in the memory 502 includes a suitable operating system (O/S) 504, compiler 505, source code 506, and one or more applications 507 in accordance with exemplary embodiments.

The application 507 comprises numerous functional components such as computational units, logic, functional units, processes, operations, virtual entities, and/or modules.

The operating system 504 controls the execution of computer programs, and provides scheduling, input-output control, file and data management, memory management, and communication control and related services.

Application 507 may be a source program, executable program (object code), script, or any other entity comprising a set of instructions to be performed. When a source program, then the program is usually translated via a compiler (such as the compiler 505), assembler, interpreter, or the like, which may or may not be included within the memory 502, so as to operate properly in connection with the operating system 504. Furthermore, the application 507 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, NET, and the like.

The I/O devices 503 may include input devices such as, for example but not limited to, a mouse, keyboard, scanner, microphone, camera, etc. Furthermore, the I/O devices 503 may also include output devices, for example but not limited to a printer, display, etc. Finally, the I/O devices 503 may further include devices that communicate both inputs and outputs, for instance but not limited to, a network interface controller (NIC) or modulator/demodulator (for accessing remote devices, other files, devices, systems, or a network), a radio frequency (RF) or other transceiver, a telephonic interface, a bridge, a router, etc. The I/O devices 503 also include components for communicating over various networks, such as the Internet or intranet.

When the computer 500 is in operation, the processor 501 is configured to execute software stored within the memory 502, to communicate data to and from the memory 502, and to generally control operations of the computer 500 pursuant to the software. The application 507 and the operating system 504 are read, in whole or in part, by the processor 501, perhaps buffered within the processor 501, and then executed.

When the application 507 is implemented in software it should be noted that the application 507 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The present invention may, for instance, be applied in the field of patient monitoring. In particular, the method 200 and apparatus 100 provided herein may be applied as an early warning for sudden deterioration of patients being monitored in a ward, and for investigation of sleep disorders. For the latter, measurements tend only to be done in a spot-check fashion when a patient is visiting a doctor. The present invention may enable continuous or semi-continuous monitoring, which may assist such investigations.

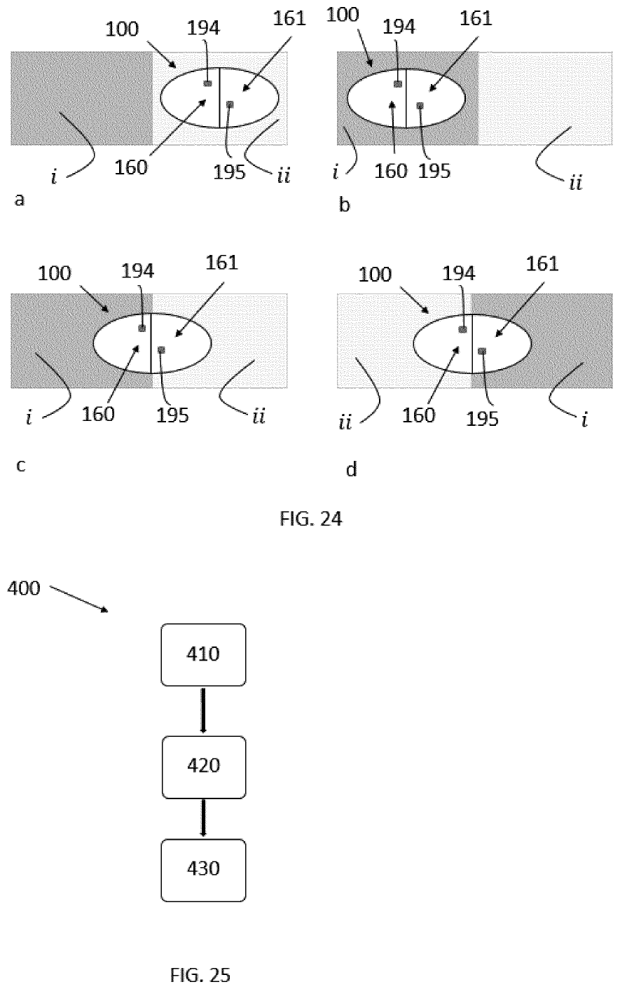

Embodiments of the present invention provide a method of positioning a sweat sensor device, e.g. a sweat analyte concentration determining apparatus as disclosed in our non-prepublished application EP 3622880 A1, in such a way, that the first part of the sweat sensor device is attached on a first skin location (i), which first skin location (i) contains apocrine sweat glands and is found in the axillae or areola or ear canal or eyelids or wings of the nostril or inguinal or perineal, or perianal regions of a mammalian subject, while the second part of the sweat sensor device is attached on a second skin location (ii) which second skin location (ii), is adjacent to the first skin location (i), namely adjacent to the axillae or areola or ear canal or eyelids or wings of the nostril or inguinal or perineal, or perianal regions of a mammalian subject, having a different sweat gland composition than the first skin location, where predominantly eccrine sweat glands are present and apocrine glands may not be present. FIG. 25 shows a flow chart illustrating the method 400 of positioning a sweat sensor device 100. The method 400 comprises:

determining 410 a first skin location (i) of a mammalian subject, which first skin location (i) contains apocrine sweat glands;

determining 420 a second skin location (ii), adjacent to the first skin location (i), and having a different sweat gland composition than the first skin location; and

positioning 430 the sweat sensor device 100 such that a first part 160 of the sweat sensor device 100 is present on the first skin location (i), while a second part 161 of the sweat sensor device 100 is present on the second skin location (ii).

The determining of the first (i) and second (ii) skin locations is achieved by detecting differences between the first (i) and second (ii) skin locations. As will be described in more detail below, such differences may be reflectivity, image of sweat on the skin, conductance, volatile organic compounds excretions, hydrophilic and hydrophobic properties, sliding resistance, skin pattern, sweat secretion behavior, and analytes uniquely excreted at the first skin location (i). Positioning of the sweat sensor device 100 to the first (i) and second (ii) skin locations may be done after determining the first skin location (i) and second skin location (ii) by means of a sweat sensor positioning device 300. Alternatively, the steps of determining the first (i) and second (ii) skin location and the step of positioning the sweat sensor device 100 may be done simultaneously, by positioning the sweat sensor device 100 on the skin and sliding it, until the first (i) and second (ii) skin locations are determined. Alternatively, the steps of determining the first (i) and second (ii) skin location and the step of positioning the sweat sensor device 100 may be done by positioning the sweat sensor device 100 on the skin and its correct positioning 430 is determined by employing parameters unique to eccrine or apocrine glands. Such parameters are uniquely excreted analytes by apocrine glands and unique sweat secretion behavior of eccrine and apocrine glands. The sweat sensor positioning device 300 and/or the sweat sensor device 100 may include a suitably programmed processor for carrying out the various method steps described below.

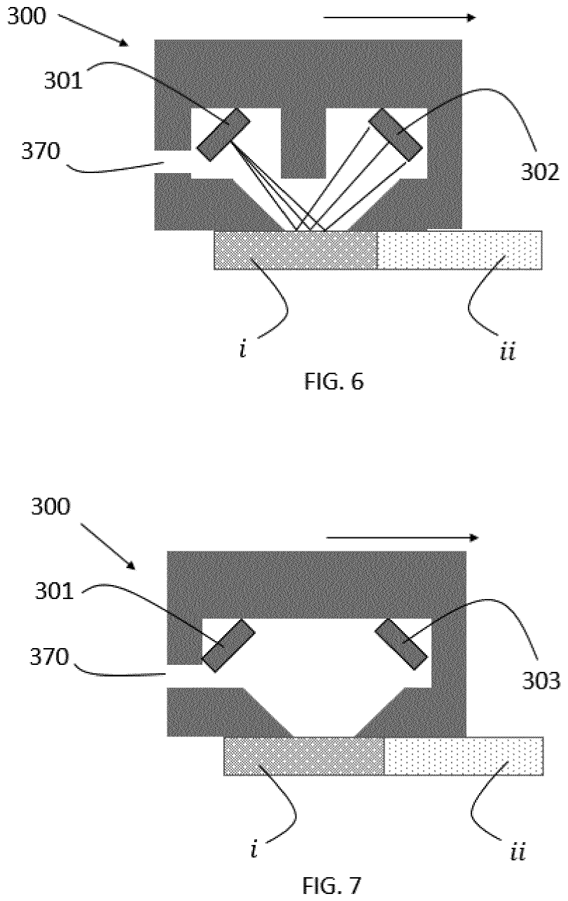

FIG. 6 illustrates a method according to an embodiment. The method comprises using a sweat sensor positioning device 300 having a light source 301 which provides light to illuminate a skin, and a light detector 302 which receives a reflection from the skin and detects reflectivity. The light source may be an artificial light source (e.g. light produced by an LED) or an opening in the housing 370 of the sweat sensor positioning device 300, which permits light to pass through the housing 370 and illuminate the skin. The light detector 302 may comprise a matrix to reconstruct an image of the skin, so skin reflectivity may be inspected in detail, resulting to increased reflectivity detection sensitivity. The first skin location (i) with apocrine sweat glands differs from the adjacent second skin location (ii), where predominantly eccrine sweat glands are present and consequently a different reflection is recorded between the said skin areas. The different skin reflection or image change is caused by sebum. First skin locations (i) contain large numbers of sebaceous glands. The apocrine glands exit into the shaft of the hair follicles and the sebaceous gland are also exiting into the same shaft. Therefore, excretions by both glands occur on the same skin location and constitute a waxy-like component, which will partially absorb light, reducing reflectivity. Since the density of hair follicles at first skin locations (i) is substantially higher than at the adjacent second skin locations (ii), sebum is excreted in higher amounts at first skin locations (i) compared to the amount of sebum excreted at the adjacent second skin locations (ii).

A user may move the sweat sensor positioning device 300 over the skin or alternatively may position the sweat sensor positioning device 300 in discrete locations on the skin and take spot measurements. Alternatively, the positioning device may move autonomously, without the need of a user moving it over the skin.

The first skin locations (i) have a first skin reflectivity, as they have a first amount of sebum on the skin, and the second skin locations (ii) have a second skin reflectivity, higher than the first skin reflectivity, as they have a second amount of sebum on the skin, less than the first amount of sebum at first skin locations (i). The ratio of the first to the second amount of sebum is lower than 1. When the sweat sensor positioning device 300 passes the interface between first i and second ii skin locations, it records a change in reflectivity. If this change of reflectivity corresponds to the first amount of sebum which is greater than the second amount of sebum, then the sweat sensor positioning device 300 determines the first skin location (i) as the location with the first skin reflection, and the second skin location (ii) as the location with the second skin reflection.

After determining the first (i) and second (ii) skin locations, the sweat sensor positioning device 300 may notify the user by providing visual (e.g. light is turned on), acoustic (e.g. a sound is played) or haptic (e.g. vibrating) feedback.

The user may then remember the first (i) and second (ii) skin locations and attach the sweat sensor device 100 to the appropriate position on the skin. Alternatively they may use a means (e.g. a marker) to mark the first (i) and second (ii) skin locations and then attach the sweat sensor device 100 to the appropriate position on the skin. Alternatively, the sweat sensor positioning device 300 may integrate a means for marking the skin on the position of the first (i) and second (ii) skin locations (e.g. by means of ink).

FIG. 7 illustrates a method according to a further embodiment. The method comprises using a sweat sensor positioning device 300 having a camera 303 to produce an image of sweat on a skin, and a light source 301 that provides light to illuminate the skin. The light source 301 may be an artificial light source (e.g. light produced by an LED) or an opening in the housing 370 of the sweat sensor positioning device 300, which permits light to pass through the housing 370 and illuminate the skin. The apocrine glands exit into the shaft of the hair follicles, so sweat will be formed at the hair follicle shaft. Eccrine glands, on the other hand, open directly onto the surface of the skin. As a user slides the sweat sensor positioning device 300 over a skin, the first skin location (i) is determined where the image shows sweat originating from hair follicles and the second skin location (ii) is the skin area before the image shows sweat originating from hair follicles.

After determining the first (i) and second (ii) skin locations, the sweat sensor positioning device 300 may notify the user by providing visual (e.g. a light is turned on), acoustic (e.g. a sound is played) or haptic (e.g. vibrating) feedback.

The user may use the sweat sensor positioning device 300 to determine the first (i) and second (ii) skin locations. Alternatively, the image records of the camera may be analyzed by image recognition algorithms and the positioning device may automatically determine the first (i) and second (ii) skin locations.

The user may then remember the first (i) and second (ii) skin locations and attach the sweat sensor device 100 to the appropriate position on the skin. Alternatively the user may use a means (e.g. a marker) to mark the first (i) and second (ii) skin locations and then attach the sweat sensor device 100 to the appropriate position on the skin. Alternatively the sweat sensor positioning device 300 may integrate a means for marking the skin on the position of the first (i) and second (ii) skin locations (e.g. by means of ink).

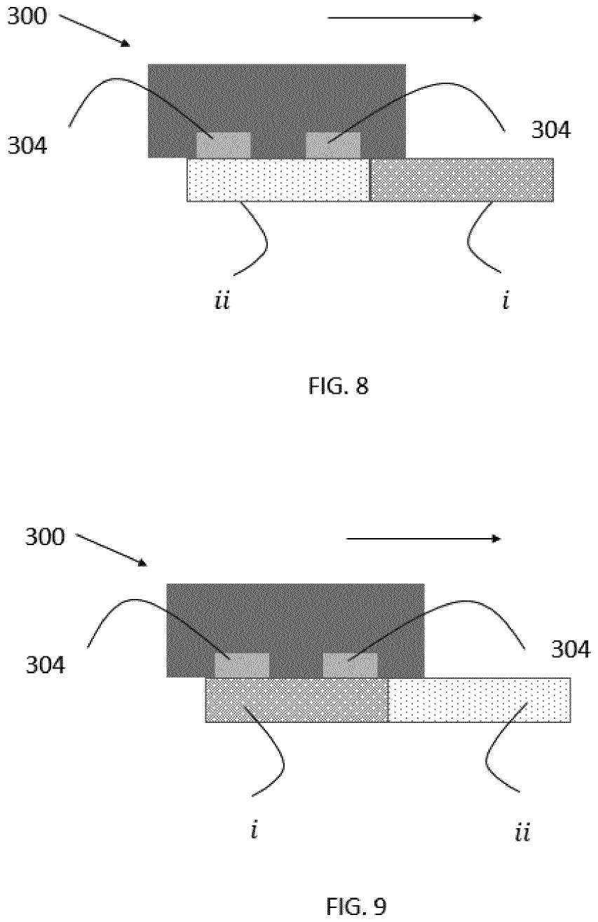

FIG. 8 and FIG. 9 illustrate a method according to a further embodiment. The method comprises using a sweat sensor positioning device 300 having a Galvanic Skin Response (GSR) sensor, and exposing two electrodes 304 of the GSR sensor in contact with a skin, which may have a typical distance of 1 cm between them, and measuring conductance between these two electrodes. Conductive gel pads may also be used to expose the electrodes to the skin, for reducing the needed time for a sufficient stable electrical conductance of the electrode-skin interface to be achieved.

Alternatively, 2 or more GSR sensors, using 2 or more pairs of electrodes, may be used, in order to improve the conductance measurement.

The measured conductance will be different between the first (i) and second (ii) skin locations, because of the different density in eccrine glands. This difference in the measured conductance should be essentially zero when the electrodes are within the first (i) and second (ii) skin locations and should only show a difference when the electrodes are positioned one in the first (i) and the other in the second (ii) skin location. A user may slide a sweat sensor positioning device 300 using a GSR sensor over the skin. Alternatively, the sweat sensor positioning device 300 may move autonomously, without the need of a user sliding it over the skin. When the GSR sensor passes the interface between the first (i) and second (ii) skin locations, it records a change in conductance, identifying the location of said skin areas.

In FIG. 8, the sweat sensor positioning device 300 using a GSR sensor is positioned in the second (ii) skin location, namely not in the axillae or areola or ear canal or eyelids or wings of the nostril or inguinal or perineal, or perianal regions of the subject and slides towards the first (i) skin location, namely the axillae or areola or ear canal or eyelids or wings of the nostril or inguinal or perineal, or perianal regions of the subject. The first skin location (i) is determined when a change in skin conductance is detected and the second skin location (ii) is the skin area before the change in skin conductance.

FIG. 9 illustrates another embodiment where a sweat sensor positioning device 300 using a GSR sensor is positioned in the first (i) skin location, namely in the axillae or areola or ear canal or eyelids or wings of the nostril or inguinal or perineal, or perianal regions of the subject and slides towards the second (ii) skin location, namely away from the axillae or areola or ear canal or eyelids or wings of the nostril or inguinal or perineal, or perianal regions of the subject. The second skin location (ii) is determined when a change in skin conductance is detected and the first skin location (i) is the skin area before the change in skin conductance.

After determining the first (i) and second (ii) skin locations, the sweat sensor positioning device 300 may notify the user by providing visual (e.g. light is turned on), acoustic (e.g. a sound is played) or haptic (e.g. vibrating) feedback.

The user may then remember the first (i) and second (ii) skin locations and attach the sweat sensor device 100 to the appropriate position on the skin. Alternatively, the user may use a means (e.g. a marker) to mark the first (i) and second (ii) skin locations and then attach the sweat sensor device 100 to the appropriate position on the skin. Alternatively, the sweat sensor positioning device 300 may integrate a means for marking the skin on the position of the first (i) and second (ii) skin locations (e.g. by means of ink).

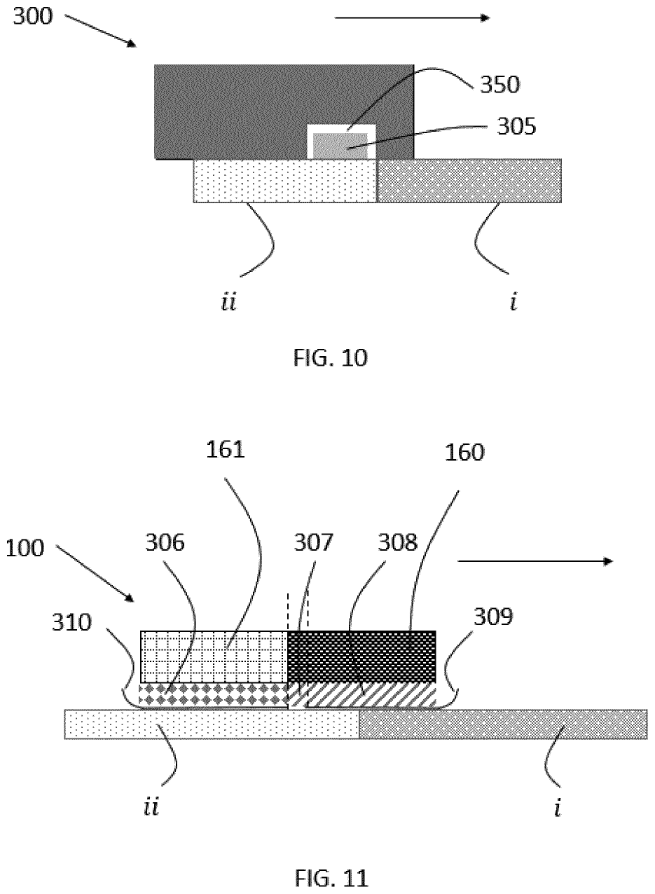

FIG. 10 illustrates a method according to a further embodiment. The method comprises detecting volatile organic compounds originating from skin locations with apocrine glands. A user may position on a skin a sweat sensor positioning device 300 using a volatile organic compound sensor 305. Such a sensor 305 may be positioned in a chamber 350 with one side open towards the skin. The sensor 305 is placed on the skin, thereby closing the chamber 350 and the volatile organic compounds remain inside it and are measured. Subsequently the chamber 350 is placed on a different skin location and again measurement is conducted. Alternatively, a user may slide the sweat positioning device 300 over the skin. In that case, the chamber 350 is preferably flushed while sliding, in order to prevent accumulation of volatile organic compounds.

A first concentration of volatile organic compounds is observed at first (i) skin locations and a second concentration of volatile organic compounds at second (ii) skin locations. The first concentration of volatile organic compounds is expected to be higher than the second concentration at the second (ii) skin location, because the population of bacteria and concentration of sebum at the first (i) skin location are higher than at the second (ii) skin location, and bacteria consume sebum, producing volatile organic compounds.

When the sweat sensor positioning device 300 is positioned at a second (ii) skin location, the second concentration of volatile organic compounds will be detected and as the sweat positioning device 300 moves to a first (i) skin location, the first concentration of volatile organic compounds will be detected, being higher than the second concentration of volatile organic compounds. Therefore the sweat positioning device 300 detects the first (i) and the second (ii) skin locations.

Vice versa, when the sweat sensor positioning device 300 is positioned in a first (i) skin location, the first concentration of volatile organic compounds will be detected and as the sweat positioning device 300 moves to a second (ii) skin location, the second concentration of volatile organic compounds will be detected, being lower than the second concentration of volatile organic compounds. Therefore the sweat positioning device 300 detects the second (i) and the first (ii) skin locations.

Alternatively, the sweat sensor positioning device 300 may use two or more volatile organic compound sensors 305. The first (i) skin location is determined where a first group of sensors detects a first concentration of volatile organic compounds and the second skin location (ii) is determined where a second group or volatile organic sensors detects a second concentration of volatile organic compounds.

The sweat sensor positioning device 300 may also move autonomously, without the need of a user sliding it over the skin.

After determining the first (i) and second (ii) skin locations, the sweat sensor positioning device 300 may notify the user by providing visual (e.g. light is turned on), acoustic (e.g. a sound is played) or haptic (e.g. vibrating) feedback.

The user may then remember the first (i) and second (ii) skin locations and attach the sweat sensor device 100 to the appropriate position on the skin. Alternatively they may use a means (e.g. a marker) to mark the first (i) and second (ii) skin locations and then attach the sweat sensor device 100 to the appropriate position on the skin. Alternatively, the sweat sensor positioning device 300 may integrate a means for marking the skin on the position of the first (i) and second (ii) skin locations (e.g. by means of ink).

FIG. 11 illustrates a method using a sweat sensor device 100, according to a further embodiment. The sweat sensor device 100 has 2 parts. A first part 160 is covered with a first adherent material 308 having hydrophilic properties, and a second part 161, is covered with any material adhering to skin 306 and it does not have hydrophilic properties. The first part 160 is designed to attach to the first (i) skin location, and the second part 161 is designed to attach to the second (ii) skin location.

The first part 160 is selected to be covered with an adherent material with hydrophilic properties 306, because as mentioned by Elkhyat et al in the book “Agache's Measuring the Skin” (Elkhyat A., Fanian F., Abdou A., Amarouch H., Humbert P. (2017) Influence of the Sebum and the Hydrolipidic Layer in Skin Wettability and Friction. In: Humbert P., Fanian F., Maibach H., Agache P. (eds) Agache's Measuring the Skin. Springer, Cham, 29 Apr. 2017, DOI https://doi.org/10.1007/978-3-319-32383-1_19, Print ISBN 978-3-319-32381-7, Online ISBN 978-3-319-32383-1) “The skin has a hydrophilic tendency on the sebaceous sites and a hydrophobic tendency on sites free of sebum. The degree of spreading of the water is a good indicator of the affinity of the skin with water. The role of the hydrolipidic film of the wettability of the skin is clearly demonstrated. The frictional behavior of the skin while contacting (touching) different materials plays a critical role in the skin's sensory perception of objects that we come into contact with”. Although sebaceous glands are distributed over the body surface, the number of sebaceous glands is different for various body locations. As said, the density of sebaceous glands is expected to be higher on the skin surface in the first (i) skin locations than on the second (ii) skin locations. Consequently, the hydrophilic/hydrophobic balance will be different for said skin surfaces.

In the field of adhesives, constantly new adhesive-compounds are developed with different properties. For instance, Adhesives Research's ARflow® proprietary hydrophilic adhesives are available as pressure-sensitive adhesives (PSAs), heat-seals and coatings.

The first 160 and second 161 parts of the sweat sensor device 100 and their adherent materials are covered with protective films. The protective film 310 of the second part 161 of the sweat sensor device 100 covers the whole area of the second part. In contrast, the protective film 309 of the first part 160 does not cover the whole area of the first part 160, and leaves a small opening, where the hydrophilic adhesive is uncovered, having a distance of preferably no more than 1 cm from the second part 161.

As the user slides the sweat device sensor device 100 over a skin, the opening leaving the hydrophilic adhesive uncovered will adhere to the first (i) skin location and therefore the first (i) skin location is determined. The second skin location (ii), is also determined and it is the skin area adjacent to the first (i) skin location. Afterwards the user may fixate the sweat sensor device 100 with a finger and peel off the 2 protective films 309, 310.

The sweat sensor device 100 may be made of flexible material and may be bent to aid the peel-off action. Sweat collection holes of the sweat sensor device should be positioned sufficiently far from the interface between the adhesives.

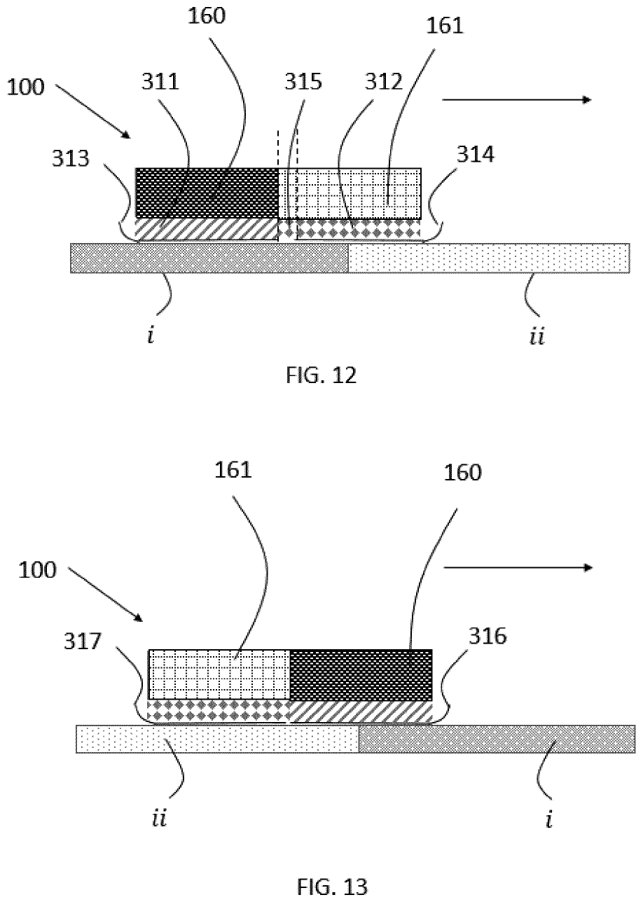

FIG. 12 illustrates a method using a sweat sensor device 100 according to a further embodiment. As in the embodiment of FIG. 11, the sweat sensor device 100 has 2 parts. A first part 160 is covered with a first adherent material 311 adhering to skin, and it does not have hydrophobic properties. A second part 161 is covered with a second adherent material 312 with hydrophobic properties. The first part 160 is designed to attach to the first (i) skin location, and the second part is designed to attach to the second (ii) skin location.

The second part 161 is covered with the adherent material 312 with hydrophobic properties, because as mentioned by Elkhyat et al in the book “Agache's Measuring the Skin” (Elkhyat A., Fanian F., Abdou A., Amarouch H., Humbert P. (2017) Influence of the Sebum and the Hydrolipidic Layer in Skin Wettability and Friction. In: Humbert P., Fanian F., Maibach H., Agache P. (eds) Agache's Measuring the Skin. Springer, Cham, 29 Apr. 2017, DOI https://doi.org/10.1007/978-3-319-32383-1_19, Print ISBN 978-3-319-32381-7, Online ISBN 978-3-319-32383-1) “The skin has a hydrophilic tendency on the sebaceous sites and a hydrophobic tendency on sites free of sebum”.

The first 160 and second 161 parts of the sweat sensor device 100 and their adherent materials 311, 312 are covered with protective films 313, 314, respectively. The protective film 313 of the first part 160 of the sweat sensor device 100 covers the whole area of the first part 161. In contrast, the protective film 314 of the second part 161 does not cover the whole area of the second part 161, and leaves a small opening, where the hydrophobic adhesive is uncovered, with a distance of maximum 1 cm from the first part 160.

As the user slides the sweat device sensor 100 on a skin, the opening leaving the hydrophobic adhesive uncovered will adhere to the second (ii) skin location and therefore the second (ii) skin location is determined. The first skin location (i), is also determined and it is the skin area adjacent to the second (ii) skin location. Afterwards the user may fixate the sweat sensor device 100 with a finger and peel off the 2 protective films 313, 314.

The sweat sensor 100 device may be made of flexible material and may be bent to aid the peel-off action. Sweat collection holes of the sweat sensor device should be positioned sufficient far from the interface between the adhesives.

FIG. 13 illustrates a method using a sweat sensor device 100 according to a further embodiment. The sweat sensor device 100 has 2 parts covered with an adhesive material. The first part 160 is covered with a first protective film 316, which provides a first sliding resistance when sliding over the first skin location (i). When the first part 160 is sliding over the first skin location (i), the said first sliding resistance can be either higher or lower than a third sliding resistance. The third sliding resistance is the sliding resistance of the first part 160 when it is sliding over second skin locations (ii). The second part 161 of the sweat sensor device 100 is covered with a second protective film 317, without any specific sliding resistance properties.

The sweat sensor device 100 may be positioned at the second (ii) skin location, namely not in the axillae or areola or ear canal or eyelids or wings of the nostril or inguinal or perineal, or perianal regions of the subject and a user may slide it towards the first (i) skin location, namely the axillae or areola or ear canal or eyelids or wings of the nostril or inguinal or perineal, or perianal regions of the subject. When the user detects a change in the sliding resistance, as the the sweat sensor device 100 is slid over the subject's skin, then the interface between the second (ii) and the first (i) skin location has been surpassed and therefore the first skin location (i) has been determined. The second skin location (ii) is the skin area before the first part 160 of the sweat sensor device 100 is subject to the first sliding resistance.

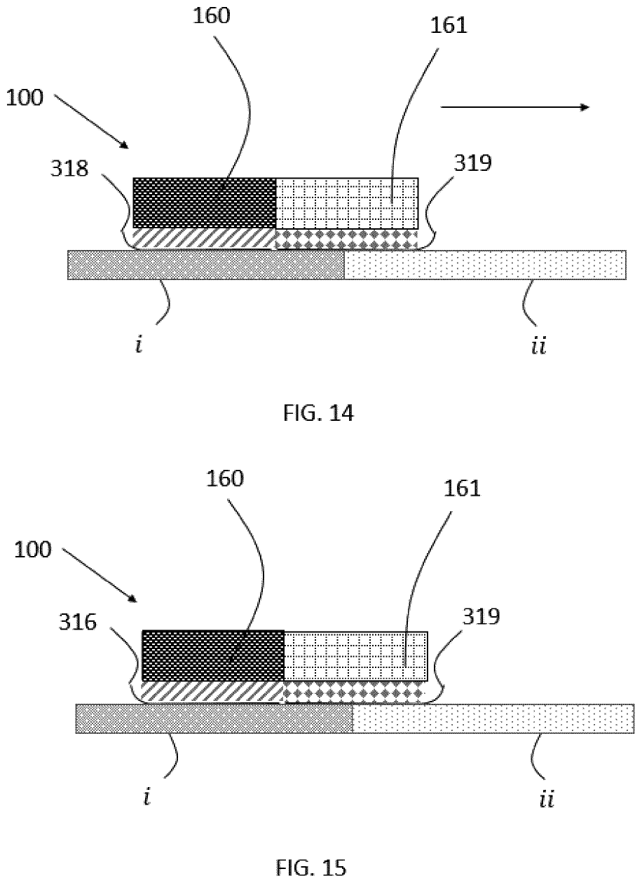

FIG. 14 illustrates a method using a sweat sensor device 100 according to a further embodiment. The sweat sensor device 100 has 2 parts covered with an adhesive material. The second part 161 is covered with a second protective film 319, which provides a second sliding resistance when sliding over the second skin location (ii). When the second part 161 is sliding over the second skin location (ii), the said second sliding resistance can be either higher or lower than a fourth sliding resistance. The fourth sliding resistance is the sliding resistance of the second part 161 when it is sliding over first skin locations (i). The first part 160 of the sweat sensor device 100 is covered with a first protective film 318, without any specific sliding resistance properties.

The sweat sensor device 100 may be positioned in the first (i) skin location, namely in the axillae or areola or ear canal or eyelids or wings of the nostril or inguinal or perineal, or perianal regions of the subject and a user may slide it towards the second (i) skin location, namely away from the axillae or areola or ear canal or eyelids or wings of the nostril or inguinal or perineal, or perianal regions of the subject. When the user detects a change in the sliding resistance, as the the sweat sensor device 100 is slid over the subject's skin, then the interface between the first (i) and the second (ii) skin location has been surpassed and therefore the second skin location (ii) has been determined. The first skin location (i) is the skin area before the second part 161 of the sweat sensor device 100 is subject to the second sliding resistance.

FIG. 15 illustrates a method using a sweat sensor device 100 according to a further embodiment. The sweat sensor device 100 has 2 parts covered with an adhesive material. The first part 160 is covered with a first protective film 316, which provides a first sliding resistance when sliding over the first skin location (i). When the first part 160 is sliding over first skin locations (i), the said first sliding resistance can be either higher or lower than a third sliding resistance. The third sliding resistance is the sliding resistance of first part 160 when it is sliding over second skin locations (ii). The second part 161 is covered with a second protective film 319, which provides a second sliding resistance when sliding over second skin locations (ii). When the second part 161 is sliding over second skin locations (ii), the said second sliding resistance can be either higher or lower than a fourth sliding resistance. The fourth sliding resistance is the sliding resistance of the second part 161 when it is sliding over first skin locations (i). Preferably when the first sliding resistance of protective film 316 is higher than the third sliding resistance, then the second sliding resistance of protective film 319 is also higher than the fourth sliding resistance. Also preferably when the first sliding resistance of protective film 316 is lower than the third sliding resistance, then the second sliding resistance of the protective film 319 is also lower than the fourth sliding resistance.

The sweat sensor device 100 may be positioned on a skin of a subject and the user slides the device over the skin. In case that the first sliding resistance is higher than the third sliding resistance, and the second sliding resistance is higher than the fourth sliding resistance, and the sweat sensor device 100 is initially positioned at a second skin location (ii), and slides towards a first skin location (i), then the user may experience overall a moderate sliding resistance because the first part 160 shows the third sliding resistance. As the sweat sensor device 100 slides and the first part 160 reaches a first skin location (i), then the user will experience overall a high sliding resistance, as the first part will show the first sliding resistance, which is higher than the third sliding resistance. If the user continues to slide the sweat sensor device 100, that far that the whole device is located at a first skin location (i), then the user will again experience overall a moderate sliding resistance because the second part 161 will show the fourth sliding resistance which is lower than the second sliding resistance. Therefore the first skin location (i) is determined where the user experiences the highest sliding resistance, which is the location on the skin where the first part 160 is subject to the first sliding resistance and the second part 161 is subject to the second sliding resistance.

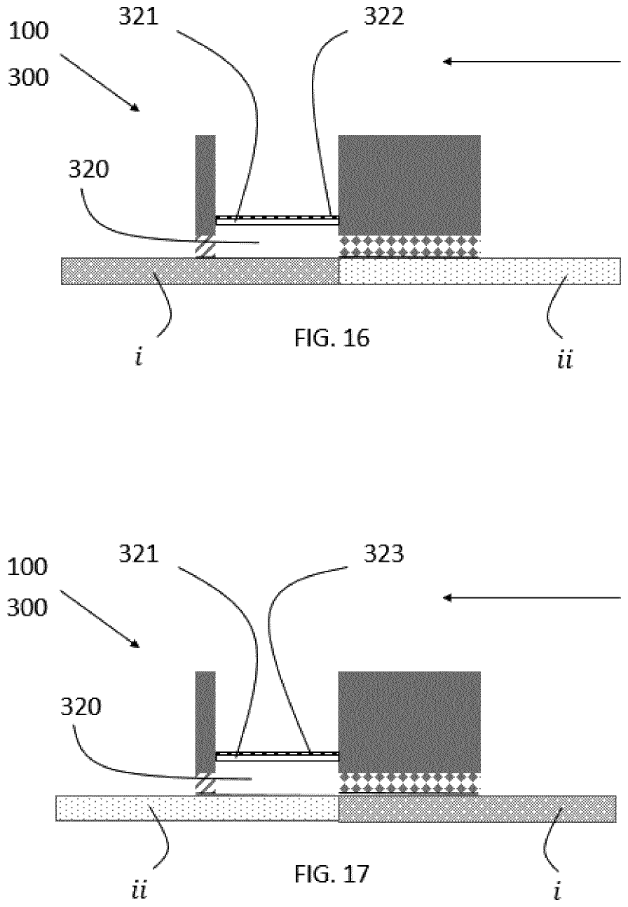

Proportionally, in case that the first sliding resistance is lower than the third sliding resistance and the second sliding resistance is lower than the fourth sliding resistance and the sweat senor is initially positioned at a second skin location (ii), and slides towards a first skin location (i), then the user may experience overall a moderate sliding resistance. This is caused because the first part 160 shows the third sliding resistance, which is higher than the first sliding resistance and the second part 161 shows the second sliding resistance which is lower than the fourth sliding resistance. As the sweat sensor device 100 slides and the first part 160 reaches a first skin location (i), then the user will experience overall a low sliding resistance, as the first part 160 will show the first sliding resistance, which is lower than the third sliding resistance and the second part 161 will still show the second sliding resistance which is lower than the fourth sliding resistance. If the user continues to slide the sweat sensor device 100, that far that the whole device is located at a first skin location (i), then the user will again experience overall a moderate sliding resistance because the second part 161 will show the fourth sliding resistance which is higher than the second sliding resistance. Therefore the first skin location (i) is determined where the user experiences the lowest sliding resistance, which is the location on the skin where the first part 160 is subject to the first sliding resistance and the second part 161 is subject to the second sliding resistance. FIGS. 16-18 illustrate a method using a sweat sensor device 100 or a sweat sensor positioning device 300 according to a further embodiment, using Moiré patterns. It is known, that when two equal regular patterns are observed in a stack, various interference patterns emerge. These interference patterns are called Moiré patterns, dependent on the angular position between these regular patterns. These Moire patterns can be observed by shining light through the stack or shining light on the stack and observe the reflection. The features recognizable in the Moiré patterns are much larger than the feature size of the actual pattern. While a feature size of about 100-300 μm is difficult to observe by eye, the much larger interference feature size is easily observable by eye. When one of the patterns is different with respect to the other, in feature size or shape, still an interference pattern will occur but of different size and shape. These Moiré patterns can also be used to detect skin features that present themselves as some kind of regular pattern. The top surface of the skin—the stratum corneum—is made out of fairly regular patch-like structures. These skin-patches are being somewhat similar to triangles with a size in the order of 100-300 μm. The size and shape of such skin-patches differ between skin areas, such as in the first (i) and the second (ii) skin locations. If a transparent foil, with a print of a similar pattern as the regular skin patch-like structure is placed on the skin, one can expect a Moiré pattern as well. The contrast of the regular patch-like skin structures should be sufficient to observe the Moiré pattern well, nevertheless there are various measures to improve the contrast:

a. Optimal angular orientation between the regular skin patch-like structure and the print of the foil.

b. Using incident light that hits the skin under a small angle that will enhance the observation of the height differences of the regular skin patch-like structures and this will enhance the contrast as well.

c. The regular structure of the print may be adapted to gender and age.

d. Apply a thin biocompatible coating to the skin to enhance reflectivity, without clogging the sweat glands.

In FIG. 16, the sweat sensor device 100 or the sweat sensor positioning device 300 has a through hole 320, which reveals a skin of the mammalian subject. A transparent foil 321 covers the through hole 320. Attached to the transparent foil there is a print with a pattern 322 reflecting a repetitive feature of a skin pattern of the first skin location (i). The user positions the sweat sensor device 100 or the sweat sensor positioning device 300 on the skin, away from the first skin location (i), namely not in the axillae or areola or ear canal or eyelids or wings of the nostril or inguinal or perineal, or perianal regions. The sweat sensor device 100 or a sweat sensor positioning device 300 is thereafter slid towards the axillae or areola or ear canal or eyelids or wings of the nostril or inguinal or perineal, or perianal regions. The first skin location (i) is determined where a Moiré pattern is observed though the through hole 320 and the second skin location (ii) is the skin area before a Moiré pattern is observed though the through hole 320.

FIG. 17 illustrates a method positioning a sweat sensor device 100, using Moiré patterns, according to a further embodiment. The sweat sensor device 100 or a sweat sensor positioning device 300 has a through hole 320, which reveals a skin of the mammalian subject. A transparent foil 321 covers the through hole 320. Attached to the transparent foil there is a print with a pattern 323 reflecting a repetitive feature of a skin pattern of the second skin location (ii). The user positions the sweat sensor device 100 or the sweat sensor positioning device 300 on a skin, on a first skin location (i), namely in the axillae or areola or ear canal or eyelids or wings of the nostril or inguinal or perineal, or perianal regions. The sweat sensor device 100 or the sweat sensor positioning device 300 is thereafter slid away from the axillae or areola or ear canal or eyelids or wings of the nostril or inguinal or perineal, or perianal regions. The second skin location (ii) is determined where a Moiré pattern is observed though the through hole 320 and the first skin location (i) is the skin area before a Moirépattern is observed though the through hole 320.

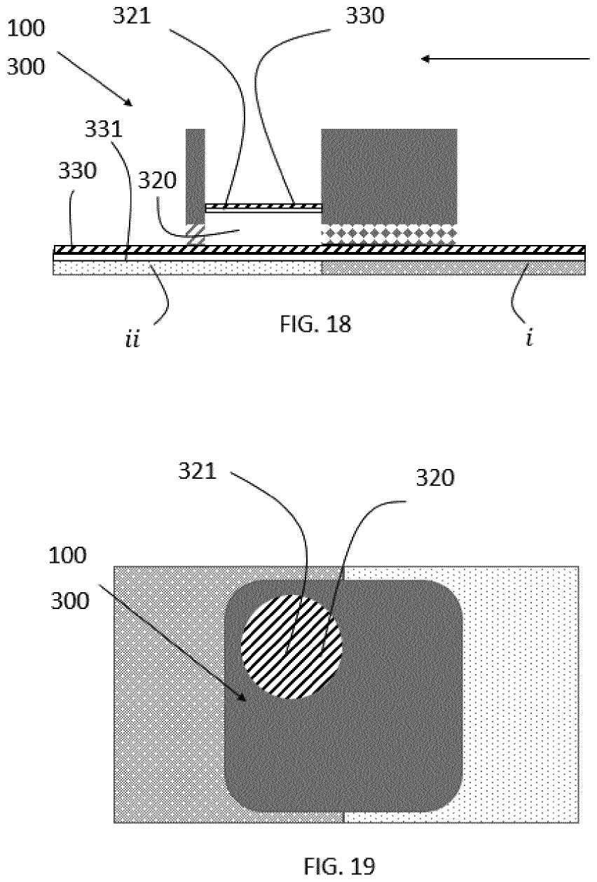

FIG. 18 illustrates a method positioning a sweat sensor device 100, using Moiré patterns, according to a further embodiment. The sweat sensor device 100 or a sweat sensor positioning device 300 has a through hole 320, which reveals a skin of the mammalian subject. A transparent foil 321 covers the through hole 320. Attached to the transparent foil is a print with a pattern 330. Another transparent foil 331 is attached to a skin of a subject. This transparent foil 331 has another print with the same pattern 330 attached.

Pattern 330 does not have to be a repetitive feature of a skin pattern as illustrated in FIGS. 16 and 17, but it can be any pattern. Preferably, the print with the pattern 330 is made of a material with a similar index of refraction as the index of refraction of sebum. The contrast of the pattern 330 attached to the transparent foil 331, which is attached to the skin, is diminished at first skin locations (i), because it is hidden by the abundance of sebum. At second skin locations (ii), lower amounts of sebum are produced, therefore this contrast is maintained.

The user moves the sweat sensor device 100 or the sweat sensor positioning device 300 on a skin. The orientation of the repetitive pattern 330 attached to the transparent foil 331 should not be parallel to the orientation of the repetitive pattern 330 attached to the transparent foil 321, in order to for a Moiré pattern to appear. As the user slides the sweat sensor device 100 or the sweat sensor positioning device 300 on a first skin location (i), no Moiré pattern appears, because sebum hides the pattern of the print attached to the transparent foil 331. When the sweat sensor device 100 or the sweat sensor positioning device 300 reaches a second skin location (ii), a Moiré pattern is observed though the through hole 320 and the second skin location (ii) is determined. The first skin location (i) is the skin area before a Moiré pattern is observed though the through hole 320.

FIG. 19 illustrates another view of the embodiments using Moiré patterns. In the top view, a through hole 320 is observed at the sweat sensor device 100 or the sweat sensor positioning device 300 and the print attached on top of a transparent foil 321 is seen. The Moiré pattern is schematically represented by a hatched circle. In reality, not a hatched but a Moiré pattern will be observed. Instead of one through hole 320, more than one through holes can be positioned at sweat sensor device 100 or the sweat sensor positioning device 300.

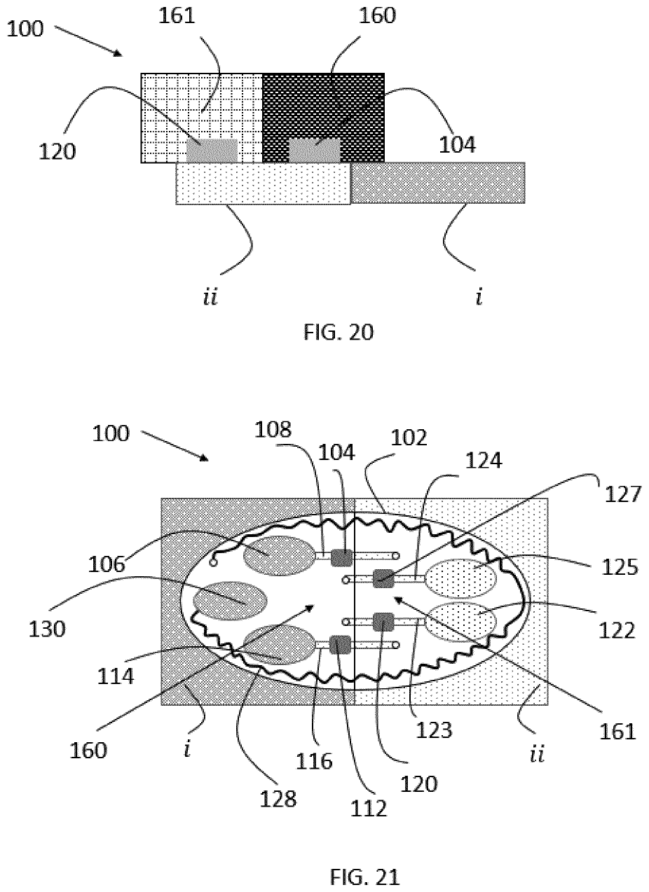

FIG. 20 illustrates a method using a sweat sensor device 100 according to a further embodiment. The method comprises detecting analytes present in apocrine sweat, which are uniquely excreted by apocrine glands. The sweat sensor device 100 has a first 160 part with a sensor 104 and a second 161 part with a sensor 120. Both sensors 104 and 120 detect analytes present in apocrine sweat. Analytes present in apocrine sweat can be apocrine secretion odor-binding proteins 1 and 2 (ASOB1 and ASOB2), carbohydrates, ferric ions, lipids, steroids, sialomucin and/or cathelicidin. The sweat sensor device 100 is placed on a skin of the patient and the correct positioning (430) of the sweat sensor device 100 is determined in the event that the sensor 104 at the first part 160 detects analytes present in apocrine sweat and the sensor 120 at the second part 161 does not detect detects analytes present in apocrine sweat.

FIG. 21 illustrates an embodiment of the configuration of the sweat sensor device 100 for detecting analytes present in apocrine sweat, which are uniquely excreted by apocrine glands. The sweat sensor device 100 comprises a single patch 102 divided into a first part 160 adhered to a first skin location (i) and a second part 161, adhered to a second skin location (ii) which is adjacent the first skin location (i). Sweat sensors 104, 112, 120 and 127 measure concentrations of analytes from sweat. Sweat is collected from collection holes 106, 114, 122 and 125 and supplies the sweat sensors via channels 108, 116, 123 and 124, respectively. Sweat is supplied to flow rate sensor 128 by sweat collection hole 130. Sensor 104 and sensor 127 determine the concentration of an analyte solely originating from apocrine glands. Sensors 112 and 120 determine the concentration of an analyte solely originating from eccrine glands. Sensor 128 is determining the total flow rate by measuring the length of the channel filled by sweat.

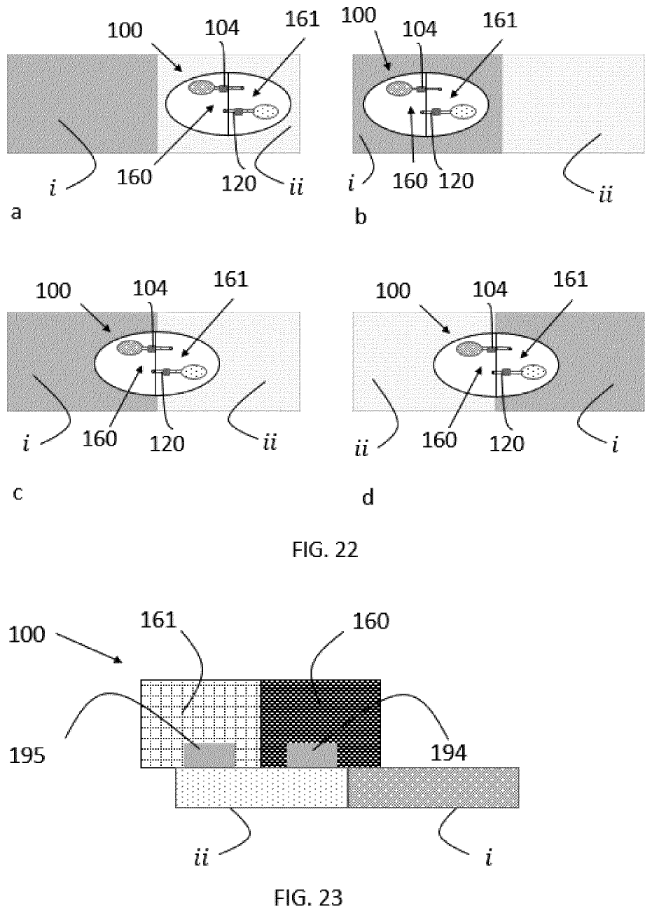

FIG. 22 illustrates four potential placements of the sweat sensor device 100 according to the embodiment of detecting analytes uniquely excreted by apocrine glands:

a. Sweat sensor device 100 is wrongly placed on a second skin location (ii) only.

b. Sweat sensor device 100 is wrongly placed on a first skin location (i) only.

c. Sweat sensor device 100 is correctly placed on a first skin location (i) and a second skin location (ii).

d. Sweat sensor device 100 is wrongly placed, where a first part 160 is positioned on a second skin location (ii), but this first part 160 of the sweat sensor device 100 was intended for a first skin location (i). Moreover, where a second part 161 of the sweat sensor device 100 is positioned on a first skin location (i), but this second part 161 was intended for a second skin location (ii).

Only when sensor 104 is detecting a concentration of an apocrine gland analyte and sensor 127 is detecting no concentration of an apocrine gland analyte, the position of the sweat sensor device 100 is correct, which is illustrated in FIG. 22c

FIG. 23 illustrates a method using a sweat sensor device 100 according to a further embodiment. The method comprises measuring unique sweat secretion behavior of eccrine and apocrine glands, as apocrine glands excrete sweat in periodic spurts while eccrine glands secrete sweat continuously. The sweat sensor device 100 has a first part 160 with a sensor 194 detecting a periodic sweat rate, and a second part 161 with a sensor 195 detecting continuous sweat rate. The sweat sensor device 100 is placed on the skin of the patient and the correct positioning 430 of the sweat sensor device 100 is determined in the event that the first sensor 194 detects the periodic sweat rate secretion and the second sensor 195 detects the continuous sweat rate secretion. If the eccrine and apocrine glands are not active simultaneously then the periodic sweat rate signal will be easily discernible. If the apocrine and eccrine glands are active simultaneously and there is overlap in their sweat secretion, then the apocrine secretion will be detected as periodic sweat rate signal on top of the continuous sweat rate signal.

FIG. 24 illustrates four potential placements of the sweat sensor device 100 according to the embodiment of measuring unique sweat secretion behavior of eccrine and apocrine glands:

a. Sweat sensor device 100 is wrongly placed on a second skin location (ii) only

b. Sweat sensor device 100 is wrongly placed on a first skin location (i) only

c. Sweat sensor device 100 is correctly placed on a first skin location (i) and a second skin location (ii).

d. Sweat sensor device 100 is wrongly placed, where a first part 160 is positioned on a second skin location (ii), but this first part 160 of the sweat sensor device 100 was intended for a first skin location (i). Moreover, where a second part 161 of the sweat sensor device 100 is positioned on a first skin location (i), but this second part 161 was intended for a second skin location (ii).

Only when sensor 194 is detecting a periodic sweat rate and sensor 195 is detecting a continuous sweat rate secretion, the position of the sweat sensor device 100 is correct, which is illustrated in FIG. 24c.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. Measures recited in mutually different dependent claims can be used advantageously combined. Any reference signs in the claims should not be construed as limiting the scope.

1. A device for assisting in the positioning of a sweat sensor, the comprising:
a processor configured to:
detect a difference between:
(a) a composition of sweat glands at a first skin location (i), wherein the composition of sweat glands at the first skin location (i) comprises apocrine and eccrine sweat glands, and
(b) a composition of sweat glands at a second skin location (ii), wherein the second skin location (ii) is adjacent to a a first skin location (i), wherein the composition of sweat glands at the second skin location (ii) comprises eccrine sweat glands and is different from the composition of sweat glands at the first skin location (i),
determine based on the difference of the composition of sweat glands at the first skin location (i) and the composition of sweat glands at the second skin location (ii), a correct position of the sweat sensor such that a first part of the sweat sensor is present on the first skin location (i), while a second part of the sweat sensor is present on the second skin location (ii).

2. The device according to claim 1, wherein the has a behavior or output that depends on whether the device is on top of the first skin location (i) and/or the second skin location (ii).

3. (canceled)

4. The device according to claim 1, wherein the processor is further configured to:
receive a measurement of a first concentration (Cₐⁱ) of a first analyte (a) in sweat excreted at the first skin location (i);
receive a measurement at least one parameter of sweat excreted by eccrine sweat glands at the second skin location (ii); and
determine a corrected concentration (Cₐ) of the first analyte (a) in sweat from the first concentration (Cₐⁱ) using said at least one parameter.

5. The device according to claim 1, wherein the device further comprises:
a light source to illuminate a skin area; and
a light detector which receives a reflection of light from the skin area.

6. The device according to claim 1, further comprising:
a light source to illuminate a skin area; and
a camera to produce an image of sweat on a skin.

7-8. (canceled)

9. The device according to claim 1, wherein the processor is further configured to:
determine the presence of volatile organic compounds;
determine the first skin location (i) in the event of detecting a first concentration of volatile organic compounds on the skin; and
determine the second skin location (ii) in the event of detecting a second concentration of volatile organic compounds on the skin, where the first concentration of volatile organic compounds is greater than the second concentration of volatile organic compounds.

10. The device according to claim 1, wherein:
the sweat sensor comprises:
a first part which is covered with a first adherent material with hydrophilic properties and a second part, which is covered with any material adhering to skin, wherein the any material-adhering to the skin does not have hydrophilic properties;
a first protective film which covers the first part, and leaves an opening of the first part of the sweat sensor uncovered;
a second protective film which covers the second part of the sweat sensor.

11. The device according to claim 1, wherein:
the sweat sensor comprise:
a first part, which is covered with any material adhering to skin and does not have hydrophobic properties;
a second part, which is covered with a second adherent material with hydrophobic properties;
a first protective film which covers the first part of the sweat sensor;
a second protective film which covers the second part of the sweat sensor, and leaving an opening of the second part of the sweat sensor uncovered.

12.-14. (canceled)

15. The device according to claim 1, wherein:
the sweat sensor comprises
a through hole, which reveals a skin of the mammalian subject;
a transparent foil that covers the through hole and comprises a pattern reflecting a repetitive feature of a skin pattern of the first skin location (i) or the second skin location (ii).

16. (canceled)

17. The device according to claim 1, wherein:
the sweat sensor comprises
a through hole, which reveals a skin of the mammalian subject; and
a transparent foil that covers the through hole and comprises a print of a repetitive pattern.

18. The device according to claim 1, wherein:
the sweat sensor comprises
a first part with a first sensor for detecting analytes present in apocrine sweat; and
a second part with a second sensor for detecting analytes present in apocrine sweat.

19. The device according to claim 1, wherein:
the sweat sensor comprises
a first part, with a first sensor for detecting a periodic sweat rate; and
a second part, with a second sensor adapted to detect a continuous sweat rate.

20. The device according to claim 5, wherein the processor is further configured to:
determine the first skin location (i) in the event of the light detector detecting a first skin reflectivity; and
determine the second skin location (i) in the event of the light detector detecting a second skin reflectivity, higher than the first skin reflectivity.

21. The device according to claim 6, wherein the processor is further configured to:
determine the first skin location (i) where the image shows sweat originating from hair follicles;
determine the second skin location (it) to be the skin area before the image shows sweat originating from hair follicles.
